(19) United States
(12) Patent Application Publication
(10) Pub. No.: US 2026/0294234 A1
(43) Pub. Date: Oct. 1, 2026

(54) FREEFORM OPTICAL COHERENCE TOMOGRAPHY BEYOND THE OPTICAL FIELD-OF-VIEW LIMIT

(71) Applicants: Northwestern University, Evanston, IL (US); The Regents of the University of California, Davis, CA (US); The Board of Trustees of the Leland Stanford Junior University, Stanford, CA (US)

(72) Inventors: Alex HUANG, Evanston, IL (US); Raymond S. FANG, Evanston, IL (US); Cheng SUN, Evanston, IL (US); Pengpeng ZHANG, Evanston, IL (US); Tingwei ZHANG, Evanston, IL (US); Hao F. ZHANG, Evanston, IL (US)

(73) Assignees: Northwestern University, Evanston, IL (US); The Regents of the University of California, Davis, CA (US); The Board of Trustees of the Leland Stanford Junior University, Stanford, CA (US)

(21) Appl. No.: 19/477,741

(22) PCT Filed: Apr. 23, 2024
(86) PCT No.: PCT/US2024/025873

§ 371 Date: Oct. 22, 2025

Related U.S. Application Data
(60) Provisional application No. 63/461,795, filed on Apr. 25, 2023.

(51) Int. Cl. A61B 3/10; A61B 3/00; G06T 7/00; G06T 7/33

(52) U.S. Cl. CPC A61B 3/102; A61B 3/0025; G06T 7/0012; G06T 7/337; G06T 2207/10101; G06T 2207/20221; G06T 2207/30041; G06T 2207/30168

(57) ABSTRACT

Imaging complex, non-planar anatomies with optical coherence tomography (OCT) is often limited by the optical field of view (FOV) achieved in a single volumetric acquisition. Systems and methods described herein provide for freeform OCT beyond the FOV limit and for the construction of compound volumetric image representing a compound imaging volume (CIV) by montaging of multiple individual OCT volumetric images. Aspects of the present disclosure include freeform robotic visible-light optical coherence tomography (vis-OCT) systems, processes for mapping a plurality of image acquisition positions based on the geometry of an object to be imaged, and processes for constructing a compound volumetric image representing a CIV from a plurality of individual OCT volumetric images acquired from a plurality of different image capture locations.

### CROSS-REFERENCE TO RELATED APPLICATIONS

This application claims the benefit of priority to U.S. Provisional Patent Application No. 63/461,795, filed Apr. 25, 2023, which is hereby incorporated by reference in its entirety.

### STATEMENT REGARDING FEDERALLY SPONSORED RESEARCH OR DEVELOPMENT

This invention was made with government support under Award Numbers R01EY030501, R01EY034740, and F30EY034033 awarded by the National Institutes of Health. The government has certain rights in the invention.

### BACKGROUND

In the past three decades, optical coherence tomography (OCT) has revolutionized fundamental investigations in various disciplines, including cardiology, dermatology, and ophthalmology. Additionally, OCT has transformed the clinical management for nearly all blinding diseases. OCT is a non-invasive microscopic imaging modality that acquires volumetric data by detecting back-scattered photons at each optical illumination position and translating the optical focus of illumination to cover the region of interest (ROI). Such a data acquisition scheme has remained largely unchanged since OCT was first reported in 1991.

### SUMMARY

The present disclosure provides systems and methods for constructing a compound volumetric image of an object to be imaged from a plurality of optical coherence tomography (OCT) volumetric images acquired by an OCT imaging system. The systems and methods involve selecting, from the plurality of OCT volumetric images, one or more overlapping pairs of OCT volumetric images. Each overlapping pair of OCT volumetric images includes a first OCT volumetric image corresponding to a first field of view (FOV) and a second OCT volumetric image corresponding to a second FOV, the first FOV and the second FOV overlapping one another in an overlap volume. The systems and methods further involve determining, for each respective selected overlapping pair of OCT volumetric images, a geometric transformation from the second FOV to the first FOV. The process by which the geometric transformations are determined involves identifying, in the first OCT volumetric image, one or more landmark volumes located in the overlap volume, identifying, in the second OCT volumetric image, the one or more landmark volumes located in the overlap volume, determining, based on a location of one or more landmark volumes in the first OCT volumetric image and a location of the one or more landmark volumes in the second OCT volumetric image, a mapping of the one or more landmark volumes in the second OCT volumetric image to the one or more landmark volumes in the first OCT volumetric image, and determining, based on the mapping, the geometric transformation from the second FOV to the first FOV. The systems and methods further involve constructing the compound volumetric image by applying the one or more geometric transformations of the one or more selected overlapping pairs of OCT volumetric images to merge the plurality of OCT volumetric images.

The present disclosure further provides systems and methods for determining a plurality of image acquisition positions for an OCT imaging system during imaging of an object to be imaged by the OCT imaging system. Each image acquisition position corresponds to a field of view (FOV). The systems and methods involve defining an object reference frame, representing, in the object reference frame, a geometry of a surface of the object to be imaged, and defining an imaging reference frame having a first axis corresponding to an axial direction of an OCT beam of the OCT imaging system and second and third axes corresponding to scanning directions of a beam scanner of the OCT imaging system. The imaging reference frame is movable relative to the object reference frame by motion of the OCT imaging system. The systems and methods further involve determining a rough positional relationship between the object reference frame and the OCT imaging system, and determining, based on the geometry of the surface of the object to be imaged in the object reference frame and the rough positional relationship between the object reference frame and the OCT imaging system, the plurality of image acquisition positions for the OCT sample arm. For each respective image acquisition position of the plurality of image acquisition positions for the OCT imaging system, the imaging reference frame is arranged such that the second and third axes form a plane that is approximately tangential to the geometry of the surface of the object in the object reference frame.

The present disclosure further provides freeform optical coherence tomography (OCT) systems. The freeform OCT systems include a light source, a reference arm, and a sample arm integrated with a robotic arm. The robotic arm is configured to move the sample arm to a plurality of image acquisition positions to provide for freeform OCT imaging. Each respective image acquisition position corresponds to a respective field of view (FOV).

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1C: Freeform imaging for expansion of the optical field of view. FIG. 1A: The optical field of view (FOV) for conventional microscopy is defined by the depth of focus (DOF) in the depth dimension and numerical aperture in the lateral dimension. The blue box represents the current optical FOV, and the yellow boxes show regions the FOV can be expanded to. To image larger structures, samples are translated along the XYZ axes. FIG. 1B: The optical FOV in OCT is determined by the optical attenuation and spectral domain sampling density in the depth direction and galvanometer angle in the lateral direction. To increase the FOV, the OCT sample arm changes its orientation relative to the sample. As it is optimal for the incident beam to be perpendicular to the sample surface, it is desirable for the OCT to travel parallel to the curvature of the sample for imaging non-planar geometries. FIG. 1C: Flexible, conformal scanning across a non-planar anatomic structure via 6 degree-of-freedom (DoF) freeform imaging.

FIGS. 2A-2H: Overview of a robotic AS-OCT system and imaging of a full outflow pathway. FIG. 2A: Schematic of vis-OCT system. CL is a collimator, FC fiber coupler, PC polarization controller, DC dispersion compensation, VA variable attenuator, TOF is a time-of-flight sensor, CMOS is a pupil camera, and M mirror. FIG. 2B: Scanning pattern of robotic AS-OCT around the limbus of the eye. The green XYZ axis is the reference frame of the eye (ERF) and the blue XYZ is the tool reference frame (TRF) of the robot. The TRF is rotated 360 degrees around the z-axes of the ERF. FIG. 2C: Magnified view of a biological sample at OCT image plane, where the OCT image plane is parallel to the major axis of Schlemm's Canal (SC) and the axis of the incident light is perpendicular to the major axis of SC. FIG. 2D: Sample arm of robotic vis-OCT, with sample arm enclosed in a white dashed square. FIG. 2E: illustration of several of the reference frames associated with the robotic arm. The FRF is the reference frame associated with the end joint of the robotic arm, and the BRF is the frame associated with the base of the robotic arm. The TRF is associated with the OCT volumetric image and the WRF is associated with the object being imaged. The WRF coincides with the ORF. The pose of the TRF relative to the FRF and the pose of the WRF relative to the BRF are defined by the user. FIG. 2F: Structural block diagram illustrating components of computer system of vis-OCT system. FIG. 2G: Flow diagram of process for constructing compound volumetric image from plurality of individual OCT volumetric images. FIG. 2H: Flow diagram of process for determining plurality of image acquisition positions for vis-OCT imaging.

FIGS. 3A-3F: Calibration of tool reference point and tool reference frame. FIG. 3A: Calibration of TCP with the focal point of OCT. Robotic OCT images target at focal point of OCT at many different positions and compute its center of rotation. FIG. 3B: En-face image of a USAF51 target card zoomed into group 1, element 2. The red and blue lines intersect the lateral position of the reference corner and correspond to the lateral center of the OCT field of view. FIG. 3C: B-scan of USAF51 card associated with the position of the blue line in FIG. 3B. The red vertical line corresponds to the lateral position of the red line in FIG. 3B. The dotted green line corresponds to the axial depth of the target card at the corner point. The scale bars are 100 μm. FIG. FIG. 3D: Calibration of TRF orientation. Angular orientation was chosen such that movement in the TRF along a specific axis results in minimal off-axis movement from the same axis in OCT volume. 3E: Translational drift of the reference point from in space as the robotic arm rotates about the TRF. FIG. 3F: Off-axis translational shifts as the robotic arm moves 200 μm in the TRF in each cartesian direction.

FIGS. 4A-4G: Image Acquisition Protocol for Outflow Pathway. FIG. 4A: Workflow for data acquisition procedure. The mouse eye was brought into the OCT field of view guided by a pupil camera. Real-time image processing was used to align the z-axis and transverse origin of the TRF and ERF. The position of the ERF origin was refined with a pre-calibration process taking the poses of the robot arm used to image the outflow pathway at 3 different positions. The WRF was set to align with the ERF and 8 OCT volumes were acquired around the eye. FIG. 4B: Overview of calibration process for imaging the limbal region of the eye, which simplifies to determining the pose of the ERF relative to the TRF. FIG. 4C: Overview of the data acquisition scheme, which involves aligning the z-axes of the ERF and TRF, pre-calibration to determine the origin of the ERF, and rotating the sample arm around the ERF z-axis. FIG. 4D: The pupil camera and real-time OCT images are used to align the lateral origins of the ERF and TRF. Optimal positioning shown in blue with suboptimal positioning in red. FIG. 4E: Real-time OCT images are used to align the z-axes of the TRF are ERF. Optimal positioning is shown in blue with suboptimal positioning in red. FIG. 4F: After the alignment of the z-axis, a pre-calibration step is used to determine the origin of the ERF. Poses used in the pre-calibration step were found by centering SC in the OCT field of view and orientating the minor axis of SC along the z-axis of the TRF. FIG. 4G: 360-degree scanning around the limbus of the eye after alignment of the WRF and ERF. Scale bars are 200 μm.

FIGS. 5A-5F: Real-time image processing used to segment cornea. FIG. 5A: Raw B-scan image in real-time. FIG. 5B: Box-filtered image. FIG. 5C: Image in FIG. 5B binarized with a low threshold. FIG. 5D: Image in FIG. 5B binarized with a high threshold. FIG. 5E: Combination of FIG. 5C and FIG. 5D minus components touching boundaries. FIG. 5F: Raw image with the top of the cornea overlayed in red.

FIGS. 6A-6K: Angle influences imaging of the outflow pathway. FIG. 6A: Schematic of SC being imaged with the OCT beam normal to the central cornea (blue arrow) and parallel to the minor axis of SC (red arrow). FIG. 6B: Schematic of the cornea being imaged with the OCT beam normal to the central cornea (blue arrow) and parallel to the minor axis of SC (red arrow). FIGS. 6C-6E: OCT of the anterior segment with OCT beam normal to the central cornea. FIG. 6C: Cross-sectional B-scan with SC in blue box. FIG. 6D: OCTA of limbal vasculature. FIG. 6E: Cross-sectional B-scan of the cornea with central cornea within the blue box and peripheral cornea within the red box. FIGS. 6F-6H: OCT of the anterior segment with OCT beam parallel to the minor axis of SC. FIG. 6F: Cross-sectional B-scan with SC in red box. FIG. 6G: OCTA of limbal vasculature. FIG. 6H: Cross-sectional B-scan of the cornea with central cornea within the blue box and peripheral cornea within the red box. FIGS. 6I-6K: CNR (FIG. 6I) between SC and surrounding tissue, (FIG. 6J) between the epithelium and stroma in the central cornea, and (FIG. 6K) between the epithelium and stroma in the peripheral cornea when the OCT beam is normal to the central cornea and parallel to the minor axis of SC. All scale bars are 100 μm.

FIG. 7: Overview of approach to montaging OCT volumes. FIG. 7A: Input data acquired during data acquisition. Poses are used to estimate the acquired volume's spatial position and determine the overlap between volumes. Each volume acquired has associated angiographic data used to determine landmark points. FIG. 7B: Steps for montaging adjacent volumes give sufficient overlap. (1) The top surface of the volumetric data is represented as a point cloud and (2) landmark points from the point cloud are extracted at vessel branch points. (3) Common landmark points between volumes are registered between each other to determine the transformation between adjacent volumes. FIG. 7C: Following the calculation of transformations between volumes, all volumes are mapped onto a common coordinate system. Each transformation is refined using a modified iterative closest point algorithm to address the loop closure problem. Scale bar for volumes is 200 μm and for angiographies is 100 μm.

FIGS. 8A-8L: SLAM for montaging of different OCT volumes. FIG. 8A: Summary of steps. FIG. 8B: Acquired B-scan. FIG. 8C: B-scan thresholded and binarized. The top eye surface of the binarized image marked in red. FIG. 8D: 3D reconstruction of binarized structure. FIG. 8E: The top eye surface of the volume represented as a point cloud. FIGS. 8F-8G: OCTA in overlapping regions. Two pairs of common vessel branch points between the OCTA shown with the blue and red circles. FIG. 8H: Montaging of adjacent surface point clouds. The point cloud of the first volume is shown in red and the point cloud of the second volume is shown in blue. FIG. 8I: Montaging of adjacent surface point clouds in only the overlapping regions. FIG. 8J: Overlapping region of 1 point cloud montaged with all overlapping regions in the eye. FIG. 8K: Point cloud representation of entire eye surface. FIG. 8L: Equaconic projection of eye, with positions along the eye represented as latitude and longitude lines. Scale bars are 100 μm.

FIGS. 9A-9E: Reconstruction of anterior segment of eye. FIGS. 9A-9B: Volumetric reconstruction of the anterior segment viewed from the (FIG. 9A) anterior and (FIG. 9B) posterior direction. The top surface of the cornea is visible from the anterior direction and the lens is visible from the posterior direction. FIG. 9C: Sample cross-section from the montaged volume, with blue arrows pointing to SC. Cross-section located at red line in FIG. 9A. FIGS. 9D-9E: Stereographic projection of (FIG. 9D) structural and (FIG. 9E) angiographic OCT volumetric data. Scale bars are 200 μm.

FIGS. 10A-10H: Robotic vis-OCT visualizes segmental patterns in SC morphology. FIG. 10A: 3D reconstruction of SC. FIG. 10B: Position of SC in context to the structural reconstruction of the eye. FIG. 10C: SC viewed along its major axis across the globe. The position of SC is overlayed in light blue. FIG. 10D: SC viewed along its minor axis. SC is a continuous hyporeflective lumen. The red arrow points to SC in the nasal and superior quadrants. Segmental patterns in SC morphology observed, with SC larger in the nasal and temporal quadrants and smallest in the superior quadrant. FIG. 10E: SC cross-sectional area, height, and width as a function of position around the globe. FIG. 10F: Relative SC volume, (FIG. 10G) height, and (FIG. 10H) width for each quadrant of the eye relative to the average value across all quadrants. Volume and height of SC largest in temporal quadrant (n=6 mouse). * indicates p<0.05, ** p<0.01, **** p<0.0001. Scale bars in FIGS. 10A-10C are 200 μm and in FIG. 10D is 100 μm.

FIGS. 11A-11C: Reconstruction of collector channels around the eye. FIG. 11A: Regional SC area and CC position for a C57BL/6 mice. The red curve represents SC area and the green lines indicate CC position. FIG. 11B: distribution of those CC per quadrant. FIG. 11C: All CC around the eye mapped. Blue arrow points to SC and green to CC. Numbering of CC corresponds to the numbering of green lines in FIG. 11A. CC labeled in red are in the nasal quadrant, green in the superior quadrant, blue in the temporal quadrant, and white in the inferior quadrant. The scale bar is 50 μm.

FIGS. 12A-12D: Resampling of Schlemm's Canal. FIG. 12A: Input data used to resample SC in the height dimension for a given volume. Volumetric OCT data and volumetric segmentation of SC are inputs to the resampling data. An OCT en face projection with the B-scan at position of the vertical dotted red line are shown in the volumetric data panel. The SC is shaded in blue in the B-scan. The scale bar for the OCT en face is 200 μm and the scale bars for the B-scan and SC segmentation are 100 μm. FIG. 12B: We determined the skeleton along the lateral positions of the segmented SC. The skeleton is shown in the orange line and the lateral positions of the SC is shaded in light blue. The scale bar for the is 200 μm. FIG. 12C: For the skeleton, we determined the normal direction to the skeleton along each point in the skeleton. The red line shows the skeleton and the orange and blue lines show the normal vectors to the skeleton. The image to the right is a zoomed in version of the red box in the left image. The scale bar on the left is 200 μm and the scale bar on the right is 50 μm. FIG. 12D: SC resampled along the skeleton with averaging along the normal direction to the skeleton. The top boundary of each B-scan was flattened to the same axial position. The scale bars are 100 μm in each direction.

FIG. 13: Resampling of Collectors Channels. FIG. 13A: Input data used to resample each individual CC from SC to distal vasculature. OCT volumetric and each CC segmentation were used as input data for the resampling. The segmentation of CC and adjacent SC in the volume are overlaid in orange. FIG. 13B: The CC segmentation was converted into a graph, with each pixel represented as a node. FIG. 13C: Example graph for a singular B-scan with the shortest path between two nodes outlined in red. FIG. 13D: The pixels corresponding to the shortest path overlaid in orange over the segmented CC and adjacent SC. FIG. 13E: The volumetric data was resampled along the lateral positions of the shortest path (path in red). Image on right is a zoomed in to the blue box on the left. FIG. 13F: CC resampled from SC to distal vasculature using path shown in red in FIG. 13E. The scale bars in FIG. 13A and FIG. 13F is 50 μm. The scale bar in FIG. 13D is 100 μm. The left scale bar in FIG. 13E is 100 μm and right scale bar is 40 μm.

FIGS. 14A-14C: Operations done to visualize entire 360 degrees of outflow pathway. FIG. 14A: The eyelid (red rectangle) blocks the outflow pathway around the nasal and temporal limbal regions of the eye. FIG. 14B: To expose the outflow pathway, small cuts in the temporal and nasal portion of the eye were made and a speculum (blue arrow) was used to expose outflow pathway. FIG. 14C: As an alternative, after small cuts were made in the temporal and nasal portion of the eye, the eye was proposed by placing sutures underneath the globe. However, proptosis of the eye causes mild blood reflux.

FIGS. 15A-15F: Real-time image processing used to segment the lens and iris. FIGS. 15A-15C: Image processing related to determining the position of the iris. FIG. 15A: Binarized B-scan minus component touching the cornea. FIG. 15B: Post-processing of binarization to isolate lens. FIG. 15C: Quadratic fit of lens position in red line and boundaries in blue lines. FIGS. 15D-15F: Image processing related to determining the position of the iris. FIG. 15D: Filter designed to detect ridges, amplifying the signal of the iris. FIG. 15E: B-scan image filtered using filter in FIG. 15D. FIG. 15F: Original B-scan with iris position overlayed in red and edge of lens given by blue lines.

FIGS. 16A-16C: Schemes for real-time alignment of axes origin between robotic sample arm and eye. FIG. 16A: The top of the fit cornea used to represent origin of eye. FIG. 16B: The center of the lens positions used to represent origin of eye. FIG. 16C: Comparison of different methods for determining eye origin, sorted from the least to most steps, compared to manual identification of origin.

FIGS. 17A-17E: Schemes for real-time alignment of optical axes between TRF and ERF. FIG. 17A: The slope of the vertical center of mass along lateral position including (1) and excluding lateral position of the lens (2) used to estimate optical axes. FIG. 17B: The slope of the vertical center of mass of the filtered image along lateral position (3), excluding lateral position of the lens (4) and excluding the cornea (5) used to estimate optical axes. FIG. 17C: The difference in center of segmented lens along left and right side of lens (6) and slope fit along lens position (7) used to estimate optical axes. FIG. 17D: The difference in average iris position to the left and right of lens (8) and slope fit along iris position (9) used to estimate optical axes. FIG. 17E: Comparison of different methods for determining eye optical axes, sorted from the least to most steps, compared to manual identification of optical axes.

FIGS. 18A-18C: Characterization of optical aberrations. FIG. 18A: Beam size at the focal plane at different wavelengths, depths, and positions. FIG. 18B: Image of distortion target grid, where the spacing between grid lines is 100 μm. FIG. 18C: Spacing between grid lines as a function of distance from the center of the OCT field of view.

FIGS. 19A-19D: Characterization of lateral resolution of robotic vis-OCT. FIG. 19A: En-Face projection of a USAF51 target card. Red arrow points to group 5, element 6. The lines of group 5, element 6 were able to be separated. FIG. 19B: Customized microfabricated phantom characterized by a three-dimensional optical profiler. The red dots have a height of 9 μm and the blue background has a height of 0 μm. FIG. 19C: En face projection of the microfabricated phantom imaged with vis-OCT. FIG. 19D: Cross-sectional B-scan of the microfabricated phantom used the determine the height of the columns. Scale bars are 100 μm.

FIGS. 20A-20I: Precision of robotic AS-OCT system and control schematics. FIG. 20A: Experimental setup for testing robotic repositioning and stability. FIG. 20B: Standard deviation and (FIG. 20C) fluctuation (range of recorded positions) of robotic arm test cube position after repositioning cube. FIG. 20D: Standard deviation and (FIG. 20E) fluctuation of test cube position when held in place. FIG. 20F: Centering algorithm applied to phantom cornea. FIG. 20G: Final recorded position of robotic arm as a function of offset from correct center position. FIG. 20H: Tilting algorithm applied to phantom lens and iris. FIG. 20I: Final recorded position of robotic arm as a function of offset from correct starting angle.

FIGS. 21A-21D: Segmental dynamic changes in SC morphology captured after pilocarpine administration. FIG. 21A: Projection of angiogram prior to and (FIG. 21B) 15 minutes after 1% pilocarpine administration. Decreased OCTA signal in the inferior region and increased signal in the nasal region were observed. The dotted green circle illustrates a region where OCTA signal decreased after pilocarpine administration and the dotted red circle illustrates a region where OCTA signal increased after pilocarpine administration, with the ability to visualize another vessel branch. Pupil size decreased following pilocarpine administration. FIG. 21C: Segmental volume within 1 degree of eye per quadrant prior to and after pilocarpine administration. Volume values averaged within 20 degrees. FIG. 21D: Volume increase as a percentage of the original volume following pilocarpine administration. Scale bars are 100 μm.

FIGS. 22A-22B: Volumetric montaging methods applied to human retina. FIG. 22 A: 3D volumetric representation of montaged human retina. Scale bars are 1 mm. FIG. 22B: Cross-sectional B-scan of montaged human retina. Scale bars are 1 mm.

### DETAILED DESCRIPTION

Imaging complex, non-planar anatomies with optical coherence tomography (OCT) is often limited by the optical field of view (FOV) achieved in a single volumetric acquisition. Specifically, limitations in mechanical scan angle and illumination orientation constrain the imaging volume for most OCT systems. The present disclosure provides systems and methods for the freeform acquisition of a plurality of OCT volumetric images and the construction of a compound volumetric image by montaging of multiple individual OCT volumetric images. In particular, aspects of the present disclosure include (i) visible-light optical coherence tomography (vis-OCT) systems capable of providing an expanded optical FOV, (ii) processes for mapping, based on the geometry of an object to be imaged (e.g. an object having a complex, non-planar anatomy such as the full anterior segment of the eye), a plurality of image capture locations for an OCT sample arm during imaging of the object to be imaged, and (iii) processes for constructing a compound volumetric image from a plurality of individual OCT volumetric images acquired from a plurality of different image capture locations.

In the following description, numerous specific details of the devices, device components, and methods of the present disclosure are set forth in order to provide a thorough explanation of the precise nature of the invention. It will be apparent, however, to those of skill in the art that the invention can be practiced without these specific details. The invention can be further understood by the following non-limiting examples.

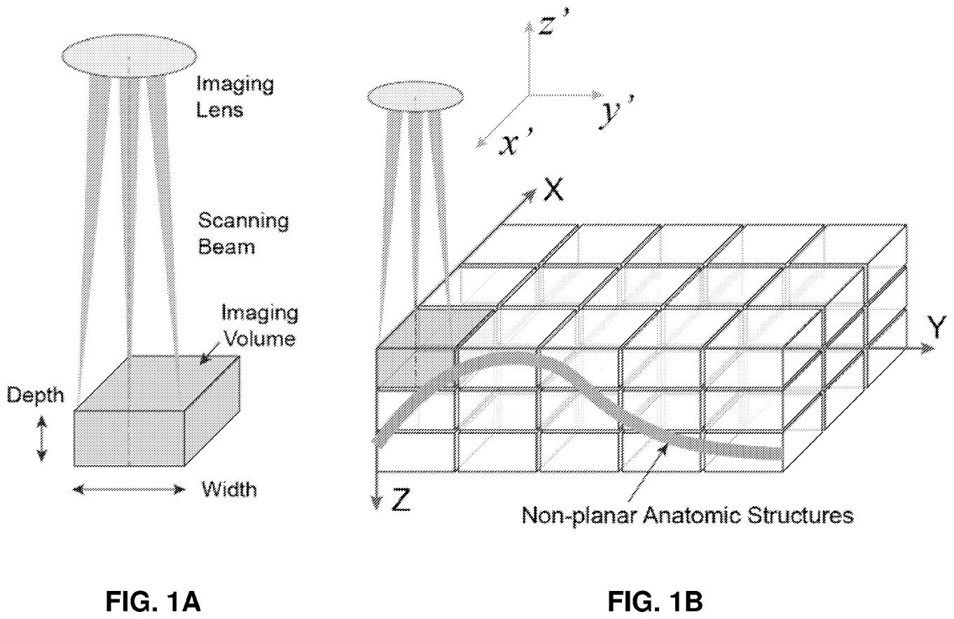

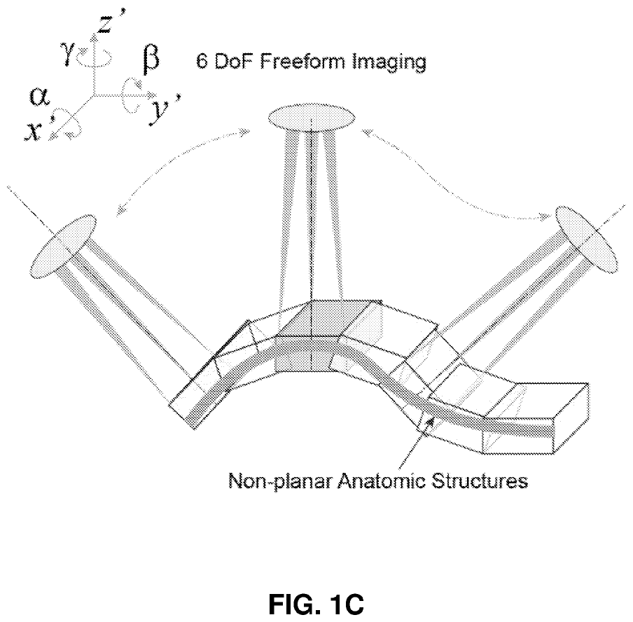

Optical imaging systems have a finite imaging volume, defining the spatial coordinates that are observable by the system. In the case of OCT, imaging volumes are determined by the field of view (FOV) and the depth of the field (DOF). As illustrated in FIG. 1A, the FOV is determined by the design of the optical system, while the DOF is governed by the optical penetration depth and spectral domain sampling density. OCT imaging volumes are often insufficient to cover large regions of interest (ROIs) with spatial heterogeneity. Extending the imaging volume is has traditionally been accomplished by 3-degree-of-freedom (DoF) translational motions in the Cartesian coordinates (FIG. 1B), as seen in nearly all modern microscopes. While such a strategy creates an enlarged compound imaging volume (CIV) suitable for stationary, flat samples, it faces several challenges in imaging non-planar anatomical structures. Foremost among these challenges is the fact that existing hybrid scanning only allows for fixed, predefined optical illumination directions. The inability to adjust the optical illumination direction can result in nonconformal imaging, in which incident illumination is oblique to the normal vector of the surface of the ROI (e.g. biological tissue). Such nonconformal imaging reduces accuracy due to degraded signal-to-noise ratio (SNR) and en-face projection artifacts. For applications involving the imaging of biological structures in vivo, such problems are particularly acute due to non-planar features such as biological structures. Furthermore, the use of 3-DoF mechanical translation to provide an enlarged CIV for non-planar sample surfaces often results in large amounts of unused data (as the CIV does not conform to the contours of the sample), resulting in both unnecessarily long data acquisition and processing times as well as unnecessarily large data storage requirements.

To simultaneously maximize the CIV and adjust the direction of incident illumination such that it remains normal to surface of the sample across the entire ROI, the present disclosure provides a freeform OCT system and processes for mapping a plurality of image capture locations for an OCT sample arm during imaging of an object to be imaged. The present disclosure provides vis-OCT systems that integrate an OCT sample arm with a six-axis robotic arm, thereby allowing for fully flexible, conformal scanning across non-planar sample geometries (FIG. 1C). Including the rotational motion capabilities of the six-axis robotic arm enhances the system's ability to provide OCT illumination in a direction normal to the sample surface, thereby optimizing imaging quality. Additionally, tailoring the conformal CIV to non-planar sample geometries effectively reduces image acquisition and processing time.

Freeform imaging, achieved, e.g., by utilizing a six-axis robotic arm, offers significant advantages but also introduces two primary technical challenges. First, the conformal CIV is specific to the sample geometry, and therefore, adjusting the direction of incident illumination such that it remains normal to the contour of the sample requires knowledge of the sample geometry's local coordinates with respect to the robotic arm's reference frames. Second, despite the high precision of the six-axis robotic arm, its accuracy is relatively limited, especially compared with the resolution that can be achieved with OCT. This discrepancy poses significant challenges in reconstructing CIVs from multiple OCT volumetric images with an axial resolution of 2-5 μm. While the most accurate commercial robots can achieve positioning precision of 5 μm, their accuracy falls behind at approximately 200 μm. Bridging this gap of nearly two orders of magnitude between spatial accuracy of such robots and the fine spatial resolution of OCT presents a formidable challenge in reconstructing CIVs. While the positioning accuracy of 6-axis robots can be improved with high-precision metrology tools, such as coordinate measurement machines and laser interferometer trackers, those approaches add unwanted burdens both in the form of technical complexity—particularly in the imaging of biological samples in vivo—and in the form of cost.

The present disclosure provides systems and methods that repurpose OCT as a built-in optical metrology tool to address the two aforementioned technical challenges. First, systems and methods of the present disclosure use OCT to delineate distinct sample geometries and provide for mapping out a plurality of image capture locations at which the sample arm is aligned such that the incident OCT beam remains conformal to surfaces of the object to be imaged. Second, systems and methods of the present disclosure use OCT to identify unique anatomical features of the sample and utilize such features to guide precise reconstruction of volumetric images over large CIVs through montaging of multiple OCT volumetric images.

To guide reconstruction of volumetric images over large CIVs, the present disclosure provides simultaneous localization and mapping (SLAM) algorithms tailored for freeform OCT. In robotics, SLAM integrates robotic positional data with photos of an unknown environment to map out unknown environments. For most applications of SLAM, robotic positional sensors are more accurate than the resolution of the objects in the environment. However, the resolution of OCT imaging is several times better than the accuracy of the robotic arm carrying the sample arm of the vis-OCT system of the present disclosure. The vis-OCT system has an axial resolution of ~1 μm while the accuracy of most precise robotic arms exceeds 100 μm. Consequently, robotic positional sensors cannot be utilized to accurately merge individual OCT volumetric images.

While the freeform robotic vis-OCT systems and corresponding methods of the present disclosure can be utilized for a variety of different imaging applications, their capabilities have been validated by in vivo imaging of the anterior segment of mouse eyes and its aqueous humor outflow (AHO) pathway. In particular, a robotic vis-OCT system according to an embodiment of the present disclosure, which has a sample arm carried by a robotic arm, was utilized to image the entire AHO pathway and its key components within a single CIV. The AHO pathway was selected for its clinical relevance to glaucoma while exhibiting anatomical complexities that make imaging with traditional OCT methods difficult. Imaging the AHO pathway presents substantial challenges when utilizing traditional OCT methods because the anatomical complexity of the pathway is defined by fine features with dimensions as minute as 2 μm, the orientation of those features is oblique to the optical axis of the eye, and the pathway has a circumferential location near the limbus. Additionally, collector channels (CCs) along with distal pathways radiate outward, extending several millimeters beyond the limbus. These distinctive anatomical characteristics underscore the importance of the capabilities of the systems and methods of the present disclosure.

As compared to typical OCT, the freeform robotic vis-OCT system of the present disclosure provides a greater degree of control in incident beam angle. The contrast-to-noise ratio (CNR) of outflow tissue and corneal layers is highly dependent on the incident angle of the OCT beam. Optimal imaging of complex non-planar tissues occurs when the incident OCT beam is conformal to the surface of the tissue. Accordingly, during the in vivo imaging of the anterior segment of the mouse eyes, a plurality of image capture locations for the OCT sample arm were mapped out, utilizing a process according to an embodiment of the present disclosure, relative to the mouse eyes in order to provide for normal illumination of the structures of interest, i.e. the AHO pathway and SC. As a result, SC was viewed with significantly higher contrast than could be achieved with fixed beam angle OCT imaging. One further advantage of freeform conformal scanning, e.g. using a robotic arm, is that the sample does not need to be rotated to achieve such normal illumination. For in vivo imaging applications, this allows for more relaxed imaging, fewer movement artifacts, and improved reproducibility.

Following the acquisition of individual OCT volumetric images from multiple poses around the mouse eyes, a process for constructing a compound volumetric image according to an embodiment of the present disclosure was utilized to integrate the plurality of individual OCT volumetric images. Existing OCT montaging algorithms register en-face projection images. However, en-face projections become nonlinearly distorted relative to each other when their curvature relative to the incident beam is significantly different from each other, as in the case of the freeform imaging of the anterior segment of mouse eyes. To circumvent this problem, the process according to the present disclosure utilized 3D volumetric surface point cloud data, which does not suffer from the same angular distortion. In particular, the process represented the surface of each volumetric image as a point cloud and utilized SLAM to integrate all of the individual volumetric images. Specifically, the process according to the present disclosure utilized both robotic positional data and acquired OCT volumetric images to montage individual volumetric images and construct a digital twin of the anterior segment of the mouse eyes. Specifically, robotic poses were used to determine if two OCT volumetric images had overlapping regions. The fine montaging of 3-D structural features was accomplished by optimizing the geometric transformations mapping the landmark points of adjacent point clouds onto each other with under 10 μm precision, an order of magnitude better than the accuracy of robotic arms. The outer surface of the merged volumes and 360-degrees reconstructed SC was smooth and continuous, indicating that the volumetric merging was accurate.

As evidenced by the imaging of the anterior segment of mouse eyes, one application for the systems and methods of the present disclosure is to improve glaucoma management. The main risk factor for glaucoma, a leading cause of irreversible blindness globally, is an elevated intraocular pressure (IOP). The equilibrium between aqueous humor inflow and outflow through the AHO pathway is crucial for IOP regulation. In both rodents and humans, the trabecular AHO pathway, comprising the anterior chamber, trabecular meshwork (TM), Schlemm's canal (SC), collector channels (CC), and distal outflow vessels, is responsible for the majority of aqueous drainage. Pathological increases in resistance within the trabecular AHO pathways lead to elevated IOP. Visualizing the AHO pathway's anatomy in three dimensions (3D) at a microscopic spatial resolution holds significant potential for revolutionizing glaucoma drug development in rodent models and enhancing the efficacy of minimally invasive glaucoma surgeries (MIGS) in patients. From a scientific perspective, reconstruction of AHO pathways can provide an improved understanding of how outflow resistance is regulated. One prevalent question is the root cause of resistance distal to SC, which may be caused by pinch points in the distal pathway.

In the imaging of the anterior segment of the mouse eyes via the systems and methods of the present disclosure, collapsed regions of the distal pathway in the resampled CC were observed, consistent with the existence of pinch points. From a surgical perspective, it has been demonstrated both experimentally and theoretically, that segmental patterns in anatomy, outflow, and CC distribution influence the success of MIGS. Systems and methods of the present disclosure thereby enable segmental differences in SC size and CC distribution to be quantified, and thus provide the technological foundation for investigating how segmental anatomical patterns can be used as biomarkers for the placement of MIGS.

According to a first aspect, the present disclosure provides a method for constructing a compound volumetric image of an object to be imaged from a plurality of optical coherence tomography (OCT) volumetric images acquired by an OCT imaging system. The method includes selecting, from the plurality of OCT volumetric images, one or more overlapping pairs of OCT volumetric images. Each overlapping pair of OCT volumetric images includes a first OCT volumetric image corresponding to a first field of view (FOV) and a second OCT volumetric image corresponding to a second FOV, the first FOV and the second FOV overlapping one another in an overlap volume. The method further includes determining, for each respective selected overlapping pair of OCT volumetric images, a geometric transformation from the second FOV to the first FOV. The process by which the geometric transformations are determined includes identifying, in the first OCT volumetric image, one or more landmark volumes located in the overlap volume, identifying, in the second OCT volumetric image, the one or more landmark volumes located in the overlap volume, determining, based on a location of one or more landmark volumes in the first OCT volumetric image and a location of the one or more landmark volumes in the second OCT volumetric image, a mapping of the one or more landmark volumes in the second OCT volumetric image to the one or more landmark volumes in the first OCT volumetric image, and determining, based on the mapping, the geometric transformation from the second FOV to the first FOV. The method further includes constructing the compound volumetric image by applying the one or more geometric transformations of the one or more selected overlapping pairs of OCT volumetric images to merge the plurality of OCT volumetric images.

In embodiments of the first aspect, the determining, from the plurality of OCT volumetric images, the one or move overlapping pairs of OCT volumetric images includes performing, for each of the one or more overlapping pairs of OCT volumetric images: comparing a first position of the OCT imaging system corresponding to the first FOV and a second position of the OCT imaging system corresponding to the second FOV, and determining, based on the comparing, that the first FOV and the second FOV overlap.

In embodiments of the first aspect, the first position of the OCT imaging system is a defined by a tool reference frame (TRF) corresponding to a first position of a robot carrying a sample arm of the OCT imaging system and the second position of the OCT imaging system is defined by a TRF corresponding to a second position of the robot.

In embodiments of the first aspect, the determining, for each respective selected overlapping pair of OCT volumetric images, the geometric transformation from the second FOV to the first FOV further includes: generating a first point cloud representing one or more surfaces of the one or more landmark volumes in the first OCT volumetric image, and generating a second point cloud representing one or more surfaces of the one or more landmark volumes in the second OCT volumetric image. In such embodiments, the determining, based on the location of the one or more landmark volumes in the first OCT volumetric image and the location of the one or more landmark volumes in the second OCT volumetric image, the mapping of the one or more landmark volumes in the second OCT volumetric image to the one or more landmark volumes in the first OCT volumetric image can include: calculating a mapping of points of the second point cloud to corresponding points of the first point cloud, wherein the calculated mapping minimizes a distance between the first point cloud and the second point cloud.

In embodiments of the first aspect, the identifying, in the first OCT volumetric image, the one or more landmark volumes located in the overlap volume comprises identifying, in the first OCT volumetric image, at least three common features of the object to be imaged that are visible in both the first OCT volumetric image and the second OCT volumetric image, and the identifying, in the second OCT volumetric image, the one or more landmark volumes located in the overlap volume comprises identifying, in the second OCT volumetric image, at least three common features of the object to be imaged that are visible in both the first OCT volumetric image and the second OCT volumetric image. In such embodiments, the first point cloud can represent outer surfaces of the at least three common features in the first OCT volumetric image, and the second point cloud can represent outer surfaces of the at least three common features in the second OCT volumetric image. Alternatively, in such embodiments, the first point cloud can represent full volumes of the at least three common features in the first OCT volumetric image, and the second point cloud can represent full volumes of the at least three common features in the second OCT volumetric image.

In embodiments of the first aspect, the method can further include, for each respective selected overlapping pair of OCT volumetric images, defining, for the first OCT volumetric image, a first tool reference frame (TRF), and defining, for the second OCT volumetric image, a second TRF, and the geometric transformation from the second FOV to the first FOV provides a pose (a translation and a rotation) of the second TRF relative to the first TRF. In such embodiment, the method can further include defining an object reference frame (ORF) providing world coordinates of the object being imaged. In such embodiments, a pose of the first TRF and a pose of the second TRF relative to the ORF can be selected based on at least one characteristic of the object to be imaged in the ORF.

In embodiments of the first aspect, the constructing the compound volumetric image by applying the one or more geometric transformations of the one or more selected overlapping pairs of OCT volumetric images to merge the plurality of OCT volumetric images can include, for at least one of the selected overlapping pairs of OCT volumetric images, transforming the second OCT volumetric image to a first tool reference frame (TRF) of the first volumetric image, and thereafter, applying a transformation mapping the first TRF to an object reference frame (ORF) to the transformed second OCT volumetric image.

In embodiments of the first aspect, the constructing the compound volumetric image by applying the one or more geometric transformations of the one or more selected overlapping pairs of OCT volumetric images to merge the plurality of OCT volumetric images can include, transforming the plurality of OCT volumetric images to a single respective reference frame defined by the object reference frame (ORF).

In embodiments of the first aspect, an image characteristic can be used to determine an origin and axes of a reference frame of the compound volumetric image, wherein the image characteristic is selected from the group consisting of an axis of symmetry, a region of interest, and a normal vector to a surface of a region of interest in the compound volumetric image.

In embodiments of the first aspect, the constructing the compound volumetric image by applying the one or more geometric transformations of the one or more selected overlapping pairs of OCT volumetric images to merge the plurality of OCT volumetric images can include: detecting errors, resulting from a loop closure problem, in an initial compound volumetric image formed by applying the one or more geometric transformations of the one or more selected overlapping pairs of OCT volumetric images, recalculating, using the detected errors, the one or more geometric transformations to provide recalculated geometric transformations, and applying the recalculated geometric transformations to merge the plurality of OCT volumetric images to construct the compound volumetric image. In such embodiments, the recalculated geometric transformations can map each of the plurality of OCT volumetric images, each of which corresponds to a respective tool reference frame (TRF), to an object reference frame (ORF) to address the loop closure problem whereby successive transformations lead to cumulative errors in the compound volume. In such embodiments, each respective recalculated geometric transformation can map a respective OCT volumetric image to the ORF to minimize a distance between respective landmark volume point clouds and all other point clouds.

In embodiments of the first aspect, the plurality of OCT volumetric images are acquired by an OCT imaging system having a sample arm configured to move with multiple translational and rotational degrees of freedom. In such embodiments, the sample arm can be carried by a robotic arm. In such embodiments, the first FOV can be determined by a first position of the robotic arm, and the second FOV can be determined by a second position of the robotic arm.

In embodiments of the first aspect, the compound volumetric image has a compound imaging volume (CIV) that is enlarged relative to an imaging volume of each respective OCT volumetric image of the plurality of OCT volumetric images.

In embodiments of the first aspect, each of the plurality of OCT volumetric images is captured from a location of the OCT imaging system at which an image plane of the OCT imaging system is approximately tangential to a surface of the object to be imaged.

In embodiments of the first aspect, a primary symmetry axis within each cross-sectional B-scan of each of the plurality of OCT volumetric images is aligned with a normal vector of a surface of the object to be imaged such that.

In embodiments of the first aspect, deviations in a center of mass in a z-axis of each respective tool reference frame (TRF) defined for a respective OCT volumetric image is minimized from both the left and right sides of a cross-sectional image.

In embodiments of the first aspect, the object to be imaged is an eye, and wherein, for an anterior segment of the eye, the reference frame of the eye is defined as the object reference frame (ORF), wherein axes of the ORF are defined using an optical axis of a respective tool reference frame (TRF) defined for a respective OCT volumetric image and parallelizing the optical axis of the respective TRF with an optical axis of the eye, the optical axis of the eye being parallel to a normal vector to a central cornea of the eye. Using the optical axis of a TRF to determine the optical axis of the eye utilizes OCT as a metrology tool helpful for the definition of the ORF. In such embodiments, the optical axis of each respective TRF can be centered on the cornea by centering a point on the cornea closest to the OCT imaging system at a center of a respective corresponding OCT field of view, and centering the OCT FOV on top of the cornea defines the origin of the ORF. In such embodiments, after identifying the optical axis and center of the eye, a predefined circular trajectory relative to the optical axis and eye center that is perpendicular to the normal vector of the eye along the scan trajectory can be mapped out.

According to a second aspect, the present disclosure provides a system for constructing an aggregate volumetric image of an object to be imaged from a plurality of optical coherence tomography (OCT) volumetric images. The system includes an OCT imaging system, which comprises a light source, a sample arm integrated with a robotic arm configured to shift a field of view (FOV) of the OCT imaging system, a reference arm, a balanced spectrometer configured to detect interference signals in light reflected from the sample arm and light reflected from the reference arm, and processing circuitry. The processing circuitry is configured to construct, based on data provided by the balanced spectrometer, a plurality of OCT volumetric images. The processor is further configured to carry out the method according to the first aspect or any embodiment thereof.

According to a third aspect, the present disclosure provides a non-transitory computer readable medium having stored thereon processor executable instructions that, when executed by one or more processors, cause the one or more processors to perform the method according to the first aspect or any embodiment thereof.

According to a fourth aspect, the present disclosure provides a method for determining a plurality of image acquisition positions for an OCT imaging system during imaging of an object to be imaged by the OCT imaging system. Each image acquisition position corresponds to a field of view (FOV). The method includes defining an object reference frame, representing, in the object reference frame, a geometry of a surface of the object to be imaged, and defining an imaging reference frame having a first axis corresponding to an axial direction of an OCT beam of the OCT imaging system and second and third axes corresponding to scanning directions of a beam scanner of the OCT imaging system. The imaging reference frame is movable relative to the object reference frame by motion of the OCT imaging system. The systems and methods further include determining a rough positional relationship between the object reference frame and the OCT imaging system, and determining, based on the geometry of the surface of the object to be imaged in the object reference frame and the rough positional relationship between the object reference frame and the OCT imaging system, the plurality of image acquisition positions for the OCT sample arm. For each respective image acquisition position of the plurality of image acquisition positions for the OCT imaging system, the imaging reference frame is arranged such that the second and third axes form a plane that is approximately tangential to the geometry of the surface of the object in the object reference frame.

In embodiments of the fourth aspect, the plurality of image acquisition positions for the OCT imaging system are determined such that each respective FOV corresponding to a respective image acquisition position overlaps with at least one other respective FOV corresponding to at least one other respective image acquisition position.

In embodiments of the fourth aspect, the defining the imaging reference frame comprises defining an origin of the imaging reference frame as a focal point of an incident beam of the OCT imaging system.

In embodiments of the fourth aspect, the method further includes controlling the OCT imaging system to move from a first of the plurality of image acquisition positions to a last of the plurality of image acquisition positions through each of the remaining image acquisition positions and to acquire, at each respective image acquisition positions of the plurality of image acquisition positions, an OCT volumetric image. In such embodiments, controlling the OCT imaging system to move from a first of the plurality of image acquisition positions to a last of the plurality of image acquisition positions through each of the remaining image acquisition positions can include providing instructions to a robotic arm, with which a sample arm of the OCT imaging system is integrated, to assume a plurality of positions that correspond to the plurality of image acquisition positions.

In embodiments of the fourth aspect, the representing, in the object reference frame, a geometry of a surface of the object to be imaged comprises identifying, in one or more OCT volumetric images obtained by the OCT imaging system, the object to be imaged or a feature of the object to be imaged. In such embodiments, the determining the rough positional relationship between the object reference frame and the OCT imaging system can include: pre-calibrating, based on the identification of the object to be imaged or the feature of the object to be imaged, the relationship between the object reference frame and the OCT imaging system, and calibrating, based on a plurality of additional OCT volumetric images obtained by the OCT imaging system, the relationship between the object reference frame and the OCT imaging system.

According to a fifth aspect, the present disclosure provides a system for constructing an aggregate volumetric image of an object to be imaged from a plurality of optical coherence tomography (OCT) volumetric images. The system includes an OCT imaging system, which comprises a light source, a sample arm integrated with a robotic arm configured to shift a field of view (FOV) of the OCT imaging system, a reference arm, a balanced spectrometer configured to detect interference signals in light reflected from the sample arm and light reflected from the reference arm, and processing circuitry. The processing circuitry is configured to carry out the method according to the fourth aspect or any embodiment thereof and/or configured to carry out the method according to the first aspect or any embodiment thereof.

According to a sixth aspect, the present disclosure provides a non-transitory computer readable medium having stored thereon processor executable instructions that, when executed by one or more processors, cause the one or more processors to perform the method according to the fourth aspect or any embodiment thereof.

According to a seventh aspect, the present disclosure provides a freeform optical coherence tomography (OCT) system. The freeform OCT system includes a light source, a reference arm, and a sample arm integrated with a robotic arm. The robotic arm is configured to move the sample arm to a plurality of image acquisition positions to provide for freeform OCT imaging. Each respective image acquisition position corresponds to a respective field of view (FOV).

Robotic vis-OCT System

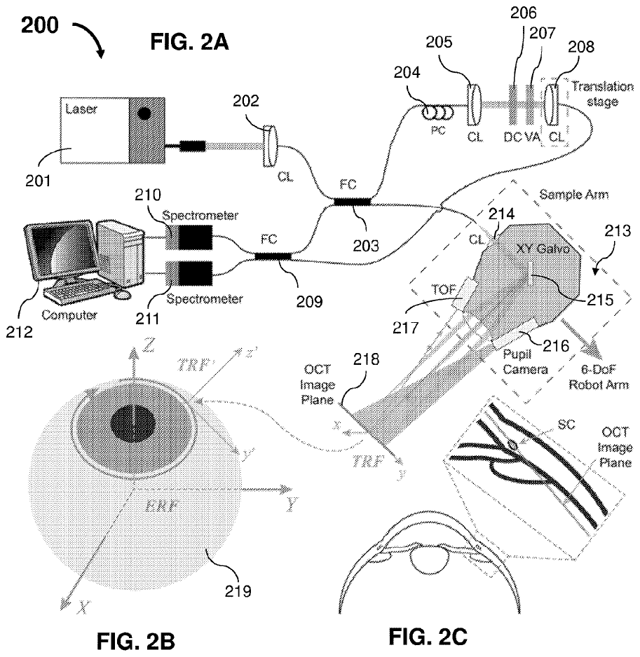

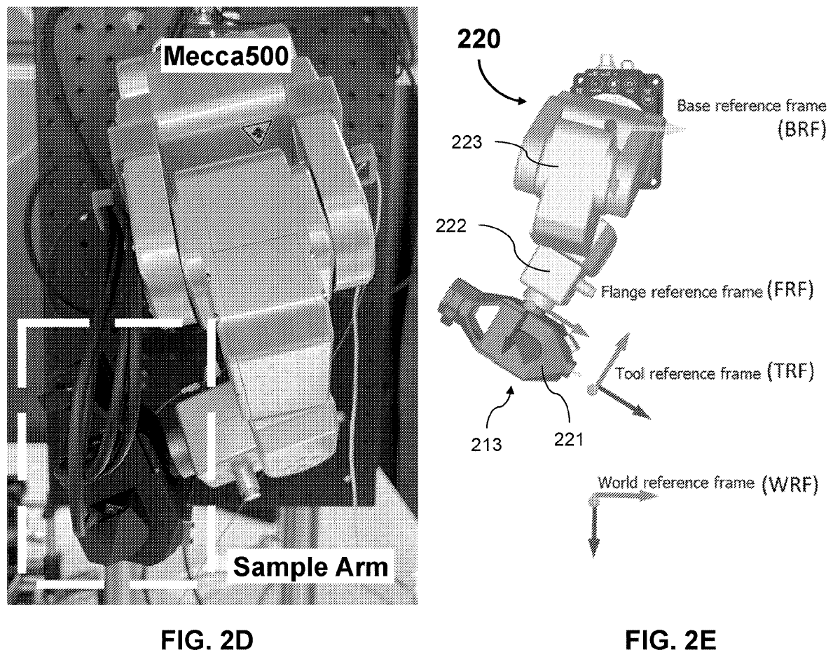

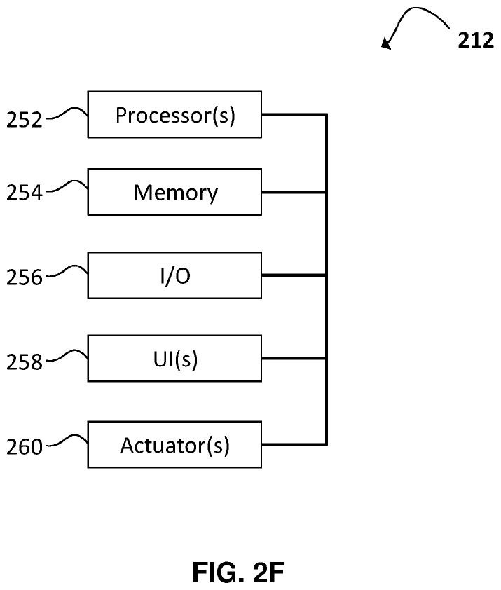

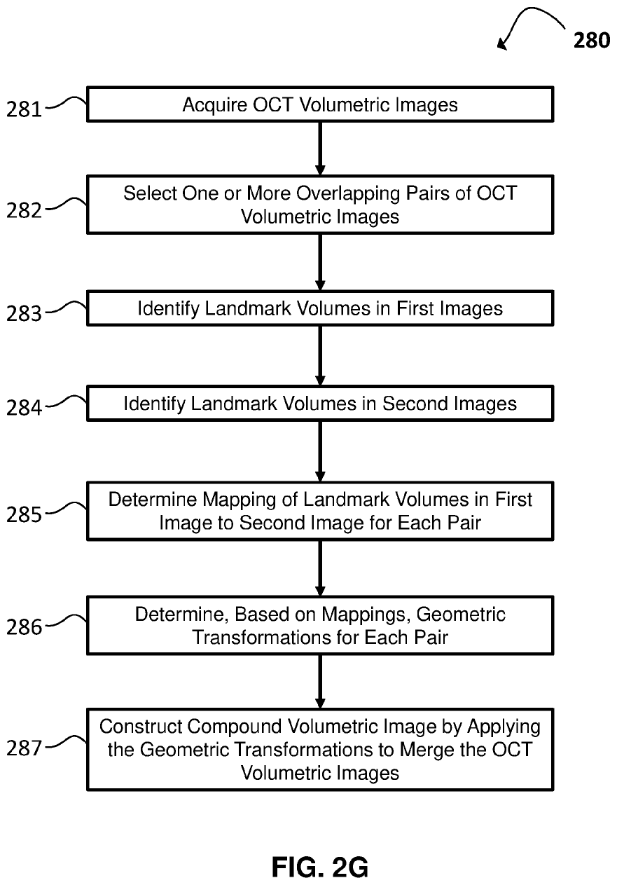

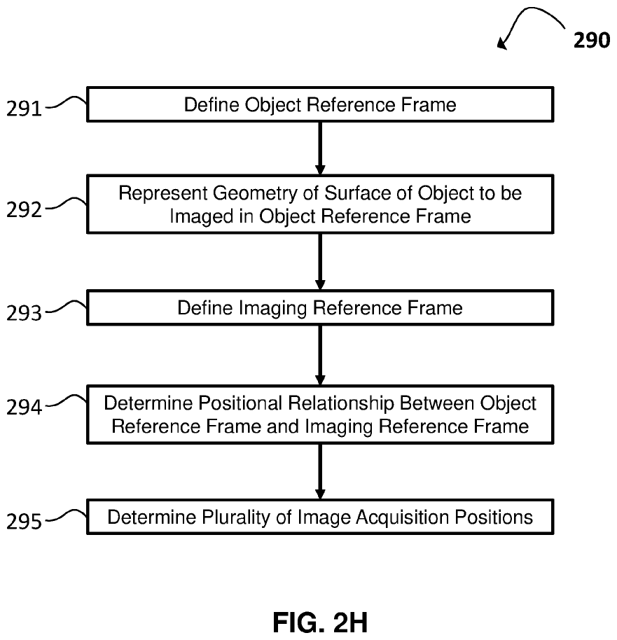

FIG. 2A illustrates a robotic visible light-optical coherence tomography (vis-OCT) imaging system 200. The configuration of the vis-OCT system 200 is illustrated in FIG. 2A. A light source, e.g. in the form of supercontinuum laser 201, provides output that is transmitted to a 90:10 fiber coupler (FC) 203 via collimator 202, with 10% sent to the sample arm 213 and 90% to the reference arm. To filter the output of the light source and minimize light outside the visible spectrum, the output can be passed through a dichroic mirror, a bandpass filter, and a spectral shaping filter. The reference arm includes a polarization controller 204, collimator 205, a dispersion compensator 206, a variable attenuator 207, and an additional collimator 208 in a translation stage. Input to the sample arm 213 from the fiber coupler 203 is collimated by collimator 214 before being scanned by a galvanometer 215 and focused onto the sample at the OCT image plane 218. Back-scattered light from the sample is passed through the fiber coupler 203 and interfered with the light from the reference arm in a 50:50 fiber coupler (FC) 209. Two well-calibrated spectrometers 210 and 211 detected the interferogram simultaneously for balanced detection. A compact sample arm 213 is mounted it on a high-precision six-axis robotic arm. A pupil camera 216 and a time-of-flight (TOF) proximity sensor 217 are integrated with the sample arm 213. The pupil camera 216 is configured to provide an initial image for locating eye 219, and the TOF sensor 217 is utilized to prevent the sample arm 213 from colliding with the sample, i.e. eye 219.

Operating the robotic vis-OCT system 200 involves managing two distinct reference frames. These frames are the six degree-of-freedom (6DoF) Cartesian coordinate system of the eyeball and the 6DoF Cartesian coordinate system of the vis-OCT imaging volume, as illustrated in FIGS. 2A and 2B. The coordinates of the sample, i.e. the eye 219, are represented using the eye reference frame (ERF) with axes XYZ, where the Z-axis aligns with the eyeball's visual axis and the origin is positioned at the center of the spherical surface approximating the cornea. The coordinates of the vis-OCT imaging volume are represented using the tool reference frame (TRF) with axes xyz. Here, the xy-plane coincides with the vis-OCT image plane 218. The orientation of x-axis and y-axis is determined by the scanning direction of the galvonmeter scanner 215. The origin of the TRF is at the center of the vis-OCT field of view, with the z-axis pointed along the optical axis of the sample arm 213. FIG. 2B specifically illustrates tool reference frame TRF′ at one point along a scanning path of the vis-OCT system 200 as it rotates by 360 degrees around the z-axis of the ERE. FIG. 2C illustrates a magnified view of the OCT image plane when it is positioned parallel to the major axis of Schlemm's Canal (SC) of eye 219 and the axis of the incident light from the sample arm 213 is perpendicular to the major axis of SC.

The embodiment of the vis-OCT system 200 can be implemented with a robotic arm 220 in the form of a 6 degree of freedom (DOF) Meca 500, produced by Mecadamic Inc., as is pictured in FIG. 2D. In FIG. 2D, the sample arm 213 is enclosed by a white dashed square. The Meca 500 supports a payload of 0.5 kg with repeatable positioning precision of 5 μm over a maximum reach of 225 mm. The sample arm is enclosed in a compact, black 3D-printed chassis 221, which is attached to the six-axis Meca500 robotic arm 220. The enclosure 221 is 3D printed using carbon fiber reinforced polycarbonate polymer materials, providing a total sample arm weight of ~0.29 kg—which is less than the specified Meca500 robotic arm payload of 0.5 kg¹. The 6 degree of freedom robotic arm 220, which additionally includes flange 222 and base 223, enables the sample arm 213 to achieve any translational (x,y,z) and angular (α, β, γ) orientation within the range of motion of the robotic arm 220.

In the vis-OCT system 200 illustrated in FIG. 2A, the sample arm 213 and the sample, i.e. the eye 219, as well as other components of the robotic arm 220 are each associated with a reference frame consisting of a 6DoF Cartesian coordinate system. Objects are capable of translating and rotating about the 3 axes of the 6DoF reference frame. The pose, which is the location and orientation of an object and its reference frame relative to another reference frame, is given by (x, y, z, α, β, γ). Under this convention, x, y, and z define the spatial offset between the origins of the reference frames. Similarly, α, β, γ refer to the relative orientation between the reference frames and follow the XYZ Euler angle convention. When describing the pose of a reference frame F₁ relative to F₀, a frame initially aligned with F₀ rotated by α degrees around the x-axis of F₀, then β degrees about the y-axis, and γ degrees about the z-axis will align its axes with F₁.

FIG. 2E illustrates multiple reference frames associated with the robotic arm 220 of the implementation pictured in FIG. 2D. A flange reference frame (FRF) is the reference frame associated with an end joint, i.e. the flange 222, of the robotic arm 220, while a base reference frame (BRF) is a frame associated with the base 223 of the robotic arm 220. The TRF is, as illustrated in FIGS. 2A-2C, associated with the OCT volumetric image acquired by the vis-OCT system 200. The WRF is a primary, static reference frame. The pose of the TRF relative to the FRF and the pose of the WRF relative to the BRF can be defined by a user. Specifically, the pose of the TRF relative to the FRF can be defined during a calibration procedure to spatially align the vis-OCT imaging volume with the TRF. The position of the WRF relative to the position of the BRF can be encoded, and therefore, the position of the TRF (which can be derived from the position of the FRF and BRF) relative to the WRF can be provided as or derived from output of the robotic arm 220. Accordingly, the WRF can be set to correspond to the ERF for convenience. Poses for the WRF, and thus the ERF, are given by XYZRPW. For the ERF, the Z-axis is the visual axis of the eye and RPW corresponds to rotations about XYZ. The origin of the ERF is the centroid of a sphere approximating the eye's corneal surface. The poses for the TRF are given by xyzrpw. For the TRF, the xy axes correspond to the scanning directions of the galvanometer and the z-axis corresponds to the primary optical axis. The origin of the TRF, known as the tool center point (TCP), is the focal point of the OCT scanning beam in the air without any galvanometer scanning.

The vis-OCT system 200 additionally includes computer system 212. FIG. 2F is a block diagram illustrating various components of the computer system 212. The computer system 212 includes one or more processors 252, memory 254, one or more input/output devices 256, one or more user interfaces 258, and one or more actuators 260.

The one or more processors 252 can include one or more distinct processors, each having one or more cores. Each of the distinct processors can have the same or different structure. The one or more processors 252 can include one or more central processing units (CPUs), one or more graphics processing units (GPUs), circuitry (e.g., application specific integrated circuits (ASICs)), digital signal processors (DSPs), and the like. The one or more processors 252 can be mounted to a common substrate or to multiple different substrates.

The one or more processors 252 are configured to perform certain functions, methods, or operations (e.g., are configured to provide for performance of functions, methods, or operations). The one or more processors 252 can perform operations embodying a function, method, or operation by, for example, executing code (e.g., interpreting scripts) stored on memory 254 and/or trafficking data through one or more ASICs. The one or more processors 252, and thus the computer system 212, can be configured to perform, automatically, functions, methods, and operations disclosed herein. Therefore, the computer system 212 can be configured to implement protocols, devices, mechanisms, systems, and methods described herein. For example, when the present disclosure states that a method or device performs task “X” (or that task “X” is performed), such a statement can be understood to disclose that computer 212 is configured to perform task “X”. The computer system 212 is configured to perform a function, method, or operation at least when the one or more processors 252 are configured to do the same.

Memory 254 can include volatile memory, non-volatile memory, and any other medium capable of storing data. Each of the volatile memory, non-volatile memory, and any other type of memory can include multiple different memory devices, located at multiple distinct locations and each having a different structure. Memory 254 can include remotely hosted (e.g., cloud) storage. Examples of memory 254 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Methods, functions, and operations described herein can be fully embodied in the form of tangible and/or non-transitory machine-readable code (e.g., interpretable scripts) saved in memory 254.

The one or more input-output devices 256 can include any component for trafficking data such as ports, antennas (i.e., transceivers), printed conductive paths, and the like. Input-output devices 256 can enable wired communication via USB®, DisplayPort®, HDMI®, Ethernet, and the like. Input-output devices 256 can enable electronic, optical, magnetic, and holographic, communication with suitable memory 254. Input-output devices 256 can enable wireless communication via WiFi®, Bluetooth®, cellular (e.g., LTE®, CDMA®, GSM®, WiMax®, NFC®), GPS, and the like. Input-output devices 256 can include wired and/or wireless communication pathways.

The one or more user interfaces 258 can include displays, physical buttons, speakers, microphones, keyboards, and the like. The one or more actuators 260 can enable the one or more processors 252 to control other components of the vis-OCT system 200. For example, the one or more actuators, which may be remotely located from the other components of the computer system 212, can be configured to control the light source 201 and the robotic arm 220.

The computer system 212, and its various components, can be distributed. For example, some components of the computer system 212 can reside in a remote hosted network service (e.g., a cloud computing environment) while other components of the computer system 212 can reside in a local computing system. The computer system 212 can have a modular design where certain modules include a plurality of the features/functions shown in FIG. 2F. For example, I/O modules can include volatile memory and one or more processors. As another example, individual processor modules can include read-only-memory and/or local caches.

FIG. 2G is a flow diagram illustrating a process 280, according to an embodiment, for generating a compound OCT volumetric image by montaging multiple OCT volumetric images. At 281, the process acquires a plurality of OCT volumetric images. The OCT volumetric images are obtained by the vis-OCT system 200. At 282, the process selects, from the plurality of OCT volumetric images, one or more overlapping pairs of OCT volumetric images. Each overlapping pair of OCT volumetric images includes a first OCT volumetric image corresponding to a first field of view (FOV) and a second OCT volumetric image corresponding to a second FOV, wherein the first FOV and the second FOV overlap in an overlap volume. The process 280 determines, at 283 through 286, a geometric transformation from the second FOV to the first FOV for each respective selected overlapping pair of OCT volumetric images. At 283, the process identifies in the first OCT volumetric image, one or more landmark volumes located in the overlap volume. At 284, the process identifies, in the second OCT volumetric image, the one or more landmark volumes located in the overlap volume. At 285, the process determines based on a location of one or more landmark volumes in the first OCT volumetric image and a location of the one or more landmark volumes in the second OCT volumetric image, a mapping of the one or more landmark volumes in the second OCT volumetric image to the one or more landmark volumes in the first OCT volumetric image. At 286, the process determines, based on the mapping, the geometric transformation from the second FOV to the first FOV. At 287, the process constructs the compound volumetric image by applying the one or more geometric transformations of the one or more selected overlapping pairs of OCT volumetric images to merge the plurality of OCT volumetric images.

FIG. 2H is a flow diagram illustrating a process 290 for determining subsequent image capture positions for an OCT sample arm based on OCT volumetric images acquired from prior capture positions. At 291, the process defines an object reference frame. At 292, the process represents, in the object reference frame, a geometry of a surface of the object to be imaged. At 293, the process defines an imaging reference frame having a first axis corresponding to an axial direction of an OCT beam of the OCT imaging system and second and third axes corresponding to scanning directions of a beam scanner of the OCT imaging system. The imaging reference frame is movable relative to the object reference frame by motion of the OCT imaging system. At 294, the process determines a rough positional relationship between the object reference frame and the OCT imaging system. At 295, the process determines, based on the geometry of the surface of the object to be imaged in the object reference frame and the rough positional relationship between the object reference frame and the OCT imaging system, the plurality of image acquisition positions for the OCT sample arm. The plurality of image acquisition positions are determined at 295 such that, for each respective image acquisition position, the imaging reference frame is arranged such that the second and third axes form a plane that is approximately tangential to the geometry of the surface of the object in the object reference frame.

### In Vivo Imaging of the Anterior Segment of Mouse Eyes

An implementation of the vis-OCT system 200 illustrated in FIG. 2A was utilized for in vivo imaging of the anterior segment of a mouse eye and its aqueous humor outflow (AHO) pathway. An implementation of the process 2E was utilized to map, based on the geometry of the anterior segment, a plurality of image capture locations for the OCT sample arm during the in vivo imaging, and an implementation of the process 2G was used to construct a compound volumetric image from the plurality of individual OCT volumetric images acquired at the plurality of different image capture locations. Specifically, following the calibration of the TRF and ERF, conformal imaging of trabecular AHO pathways was performed. This was achieved by rotating the TRF 60 degrees about either the ERF X or Y-axes, which is near the angle of the trabecular AHO pathway relative to the ERF. Following this initial rotation, the OCT sample arm was adjusted to rotate the TRF 360 degrees around the ERF's Z-axis (path shown by the trajectory in FIG. 2B). For each sample arm position, the scanning beam of the OCT is conformal to the structure being imaged, with the z-axis of the TRF normal to the tissue surface (FIG. 2C). Conceptualizing SC as an ellipse, this aligned the minor axis of SC with the axial direction of the OCT reference frame, enabling maximization of the axial resolution for imaging SC.

### Calibration of Tool Reference Frame

To accurately define the sample geometry, i.e. by defining the ERF coordinate system, relative to the TRF, a calibration process was implemented. The calibration process involved matching the TRF coordinate system with the OCT imaging volume. First, the origin, i.e. the tool center point (TCP), of the TRF was calibrated as the focal point of the vis-OCT system without galvanometer scanning. For this process, a stationary target card was utilized.

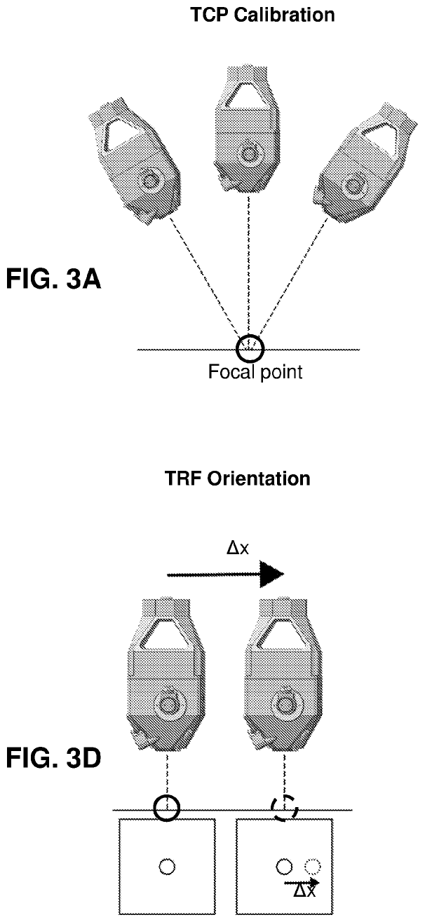

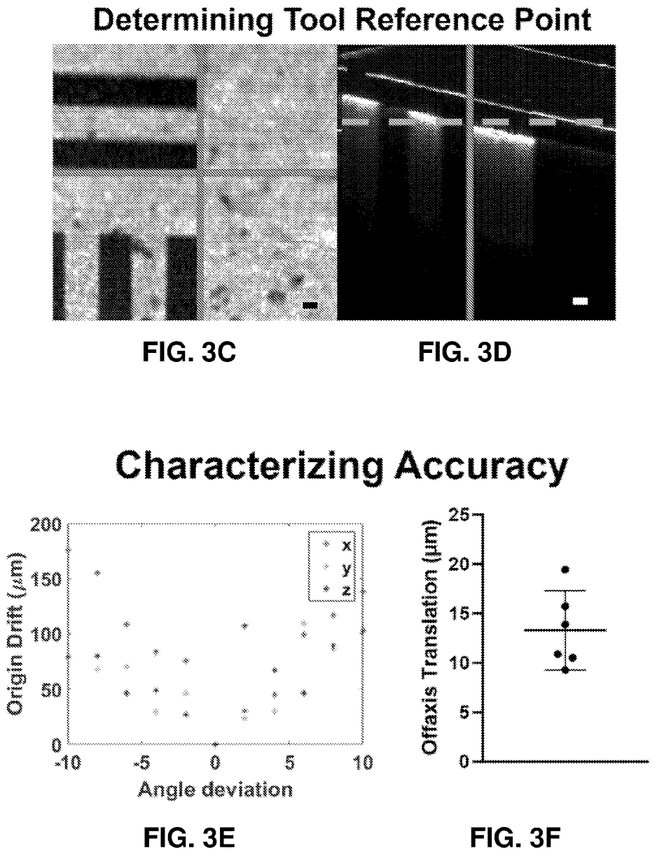

To calibrate the TCP, which is the origin of the TRF, with the focal point of our vis-OCT system without galvanometer scanning, a stationary target point was imaged at the focal plane and the center of the OCT FOV at multiple robot arm orientations (FIG. 3A). Following the collection of the robot joint angles used to image the target point, the “Define TCP” tool of the commercial RoboDK software was utilized to determine the point about which the robot arm was rotating around, which is the focal point of the robotic vis-OCT. To ensure that the stationary target point was located at the vis-OCT focal point, the sample arm position was adjusted to maximize the OCT signal returning from a mirror without galvanometer scanning and set the target point to have the same position as the mirror. A reference corner of a fixed rectangle on a plastic USAF51 target card attached to the top of a piece of paper was utilized as a stationary target point (FIG. 3B). The robot arm was moved until the reference corner was at the lateral center of the OCT field of view (intersection of vertical and horizontal lines in FIG. 3B). Next, the robot arm was adjusted such that the surface of the reference corner (a-line given by the vertical line in FIG. 3C) was set to a fixed axial position corresponding to the depth of the OCT focal plane (dotted line in FIG. 3C). This process was repeated multiple times, where the USAF51 target card was held stationary and the robotic arm moved around the stationary target point. For each imaging configuration, the joint positions of the robotic arm were recorded. After imaging with four different poses, the corresponding joint positions were input into a software tool (specifically, the “Define TCP” tool of the RoboDK software) to determine the spatial location of the point that the robot arm moves around, which, in this case, is the stationary target point. After determining the TCP using the 4 poses, an additional pose was added and the TCP was recalculated. Additional poses were added until the position of the TCP converged, i.e. shifted by less than 0.1 mm with the addition of another joint position. A total of 6 points was required prior to convergence of the TCP.

To spatially align the TCP and the focal point of the vis-OCT in air, a stationary target point was imaged at a fixed position in the OCT image plane at different robotic poses. In principle, the stationary target point could be any object with sharp features. To ensure the stationary target point was located at the OCT focal point in the axial dimension, a mirror was added to the sample arm and the position of the sample arm was adjusted to maximize a signal from the mirror. The axial position of the mirror in the reconstructed OCT volume was taken as the axial position of the vis-OCT focal point.

To orient the z-axis of the TRF along the primary optical axis of the OCT and the x and y-axes along the scanning directions of the OCT, deviations between the relative angular orientation of the OCT image volume and the current TRF were minimized. An initial estimate for the relative pose of the OCT image volume to the FRF was taken based on the mechanical design of the sample arm, and the relative orientation of the TRF to the FRF was set according to this estimate. For a specified cartesian axis direction, the robotic arm position in the TRF was translated by 500 μm while imaging the stationary target point. After each translation, the difference in the target point position within the OCT imaging volume was measured by comparing the target point position within the OCT imaging volume before and after the movement (FIG. 3D). After each measurement, the orientation of the TRF relative to the FRF was changed in increments of 0.5 degrees along the axes perpendicular to the movement axis until the off-axis distance the target point moved after the translation was minimized. Once the distance was minimized, the relative orientation between the OCT image volume and TRF along the specified axis was optimized. This process was repeated for all axes.

To assess the accuracy of the TCP calibration, the USAF51 corner reference point was positioned at the TCP and the sample arm was rotated around that point to confirm accurate calibration (if the TCP is accurately calibrated correctly, the reference point remains at a fixed position in the OCT volume regardless of any rotation). Specifically, the TRF was rotated by +10 and −10 degrees about the x, y, and z-axis of the TRF in increments of 2 degrees (FIG. 3E). Following each rotation, the position of the reference corner point was tracked. The TCP shifted by less than 200 μm for every rotational orientation, which was well within the positional accuracy of the robot arm. To assess the calibration of the axes directions, the robot arm was translated 200 μm in the +z, −z, +y, −y, +x, and −x directions. Following each translation, spatial coordinates of the reference point relative to the original position with no translation were recorded. The off-axis translation, defined as translational changes in the reference point location along the axes that were not translated in the TRF but translated in the OCT image plane, was 13.3±3.8 μm (FIG. 3F). This off-axis deviation was not statistically significant from the lateral resolution of the OCT system, which was about 8 μm.

### Image Acquisition Process for Outflow Pathway

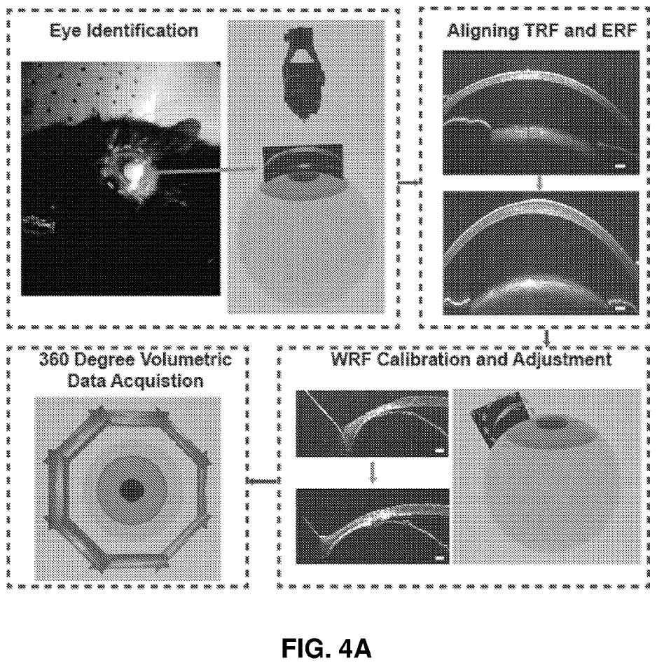

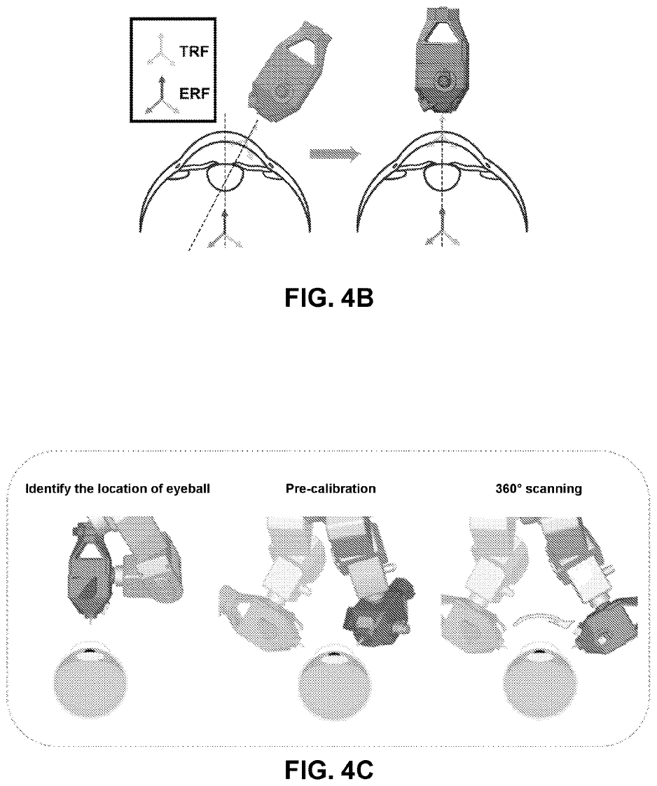

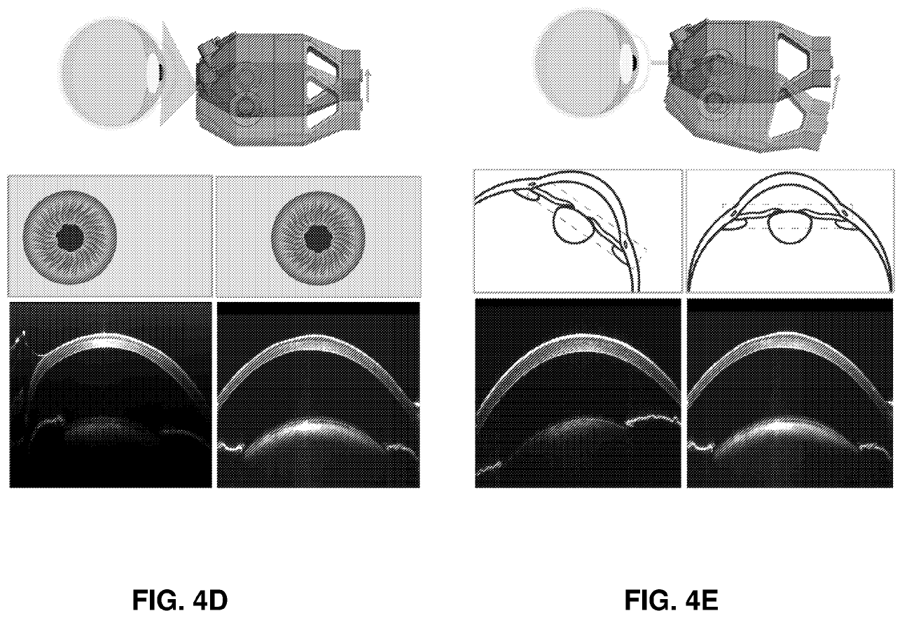

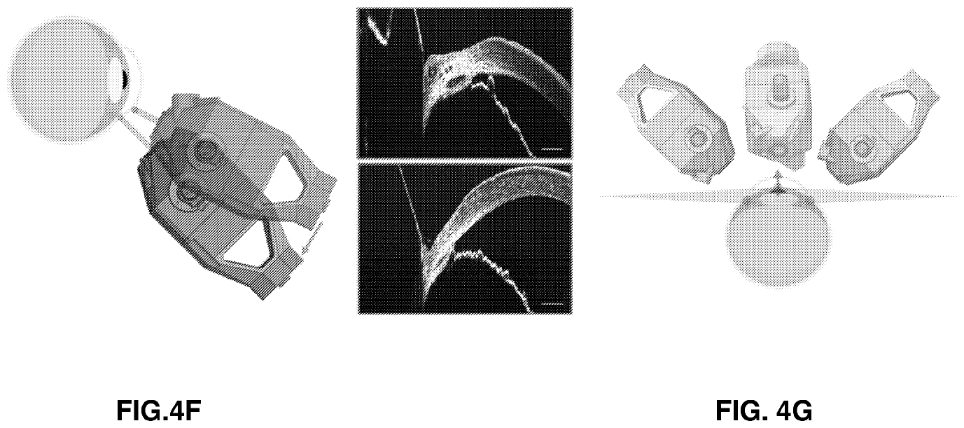

To image the entire AHO pathway, the TRF was rotated about the z-axis of the ERF to provide 360 degree volumetric data acquisition. Prior to 360 degree volumetric data acquisition (FIG. 4A), the optical axis of the eye was identified, the TRF and ERF were aligned, and the WRF was calibrated and adjusted (FIG. 4B).

To determine a trajectory for the robotic OCT around the limbus of the eye, an important step was to determine the pose of the ERF coordinate system relative to the TRF. To calibrate the pose of the ERF coordinate system relative to the TRF, the optical axis of the eye was identified, the origin of the ERF was estimated (i.e. “pre-calibration” was performed), and data was acquired while scanning 360 degrees around the outflow pathway (FIG. 4C). Additional details of the process by which the pose of the ERF coordinate system relative to the TRF was calibrated are provided below.

Step 1: Bring the central cornea of the mouse into the OCT imaging volume FOV. At this point, real time B-scans were acquired along the x and y-axes of the TRF for subsequent image analysis. These scans can be acquired manually, although it is possible to do so automatically with a pupil camera. For example the position of the pupil in the pupil camera can be identified, the offset of the pupil position to the center of the FOV can be calculated, and the TRF can be translated by the offset. The TOF sensor can then be used to translate the TRF z-axis until the mouse cornea is in focus. During this process, the position of the eye relative to the environment was documented. As the directions of the X and Y axes are determined relative to the Z-axis of the ERF, documenting the position of the eye relative to the environment allows for determination of the orientation of the quadrants of the eye relative to the X and Y-axes of the ERF.

Step 2: Parallelize the z-axis of the TRF and the Z-axis of the ERF (aligning the pose of the TRF such that R and P angles are zero relative to the ERF). This is equivalent to aligning the pose of the TRF such that the xy-plane of the TRF is parallel to the XY-plane of the ERF. For input data, real-time B-scans along the xz-plane and yz-plane in the TRF are analyzed. Using the hypothesis that the primary symmetry axis within each of the cornea B-scan images closely resembles the Z-axis in ERF, the symmetry axis in each B-scan was parallelized with the optical axis of the TRF. This was accomplished with the following process:

(A) The relative orientation of the z-axis of the TRF and the Z-axis of the ERF were evaluated along a given axis using the slope of a line fit to the center of mass in the axial direction as a function of transverse position x. The center of mass was calculated as a function of position x with the following equation where I(z,x) is the intensity of the central B-scan at depth z and position x:

$${COM}_{y} ( x ) = \frac{{\sum }_{z} I ( z , x ) z}{{\sum }_{z} I ( z , x )}$$

(B) After COM_{y}(x) was calculated, a line was fit to COM_{y}(x) and the fitted slope was used as an estimate for relative alignment of the z-axis of the TRF and Z-axis ERF. When the z-axes of the TRF and ERF are well aligned, the fitted slope has a value near zero. The greater the misalignment between the z-axes, the higher the absolute value of the fitted slope, with the sign of the slope indicating whether the z-axis of the TRF was aligned clockwise or counterclockwise to the Z-axis of the ERF along a given scan axis.

(C) Based on the slope of a line fit to COM_{y}(x), the sample arm was rotated around the corresponding axis in the TRF by angle p*slope(COM_{y}(x)), where p is a constant determining the rate of alignment. The corresponding axis is the x-axis for the fast-scanning axis B-scan and the y-axis for the alternate axis B-scan. This process adjusts the R and P angle of the TRF pose relative to the ERF. This process was continued until the relative orientation is satisfactory.

(D) Although COM_{y}(x) is curved and not strictly linear, testing on a mouse eye and anterior segment eye phantom found this method to be simpler and more reliable as compared to the relative position of the iris on both sides of the B-scan and tilt of the lens. Fitting a line could be considered a first-order Taylor approximation of COM_{y}(x). High correlation between the value of the slope of COM_{y}(x) with manual lines drawn normal to the lens was found (correlation of 0.967 for hundreds of images).

(E) FIG. 4D illustrates the process of parallelizing the z-axis of the TRF and Z-axis of the ERF. The red and blue shaded sample arms in the top panel correspond to poses where the z-axis of the TRF is parallel and oblique to the Z-axis of the ERF respectively. Representative B-scans where the z-axis of the TRF and the Z-axis are parallel and oblique to each other are shown underneath the red and blue boxes.

Step 3: Spatially overlapping the z-axis of the TRF with the Z-axis in ERF. This process aligns the x and y origin of the TRF with the X and Y origin of the ERF. To determine the X and Y origin of the ERF, the apex of the cornea is located in the B-scans (see Real-Time Corneal Apex Segmentation, described infra) and the TRF is translated accordingly.

To segment the cornea, the following process was performed. A central B-scan was acquired in real-time and mean-filtered to increase signal quality. The original image was sized 1024×128 and the mean filter had size 9×3. For faster processing, the image was downsampled by a factor of 8 in the axial direction, at which point the preview image was sized 128×128. The cornea was automatically segmented using a hysteresis thresholding scheme. The image was binarized with the application of an automatically defined high threshold (85th percentile of image intensity) and a low threshold (65th percentile). Pixels with intensity above the low threshold but below the high threshold connected to the border were removed to eliminate any autocorrelation component. The largest connected region with intensity above the low threshold but not removed was considered the structure of the eye. A second-degree polynomial was fit to the top of the binarized image to identify the top surface of the cornea. The lateral position of the apex of the fitted cornea was used to estimate the origin of the ERF along the scanning direction. Based on the offset between the apex and central A-line, the TRF was translated by the corresponding spatial distance. This was repeated until user satisfaction. At this point, the position of the real-time segmented cornea was 1.5 mm (roughly the radius of the reconstructed eyes) above the origin of the ERF in the Z-axis of the ERF.

In identifying the optical axis of the eye, the lateral origin of the ERF (FIG. 4D) was first determined. In FIG. 4D, the sample arm shaded in red demonstrates a position where the origin of the TRF is spatially offset from the origin of the ERF in the lateral direction. The position of the eye and the central B-scan corresponding to the offset sample arm position are found below in the red boxes. In the red boxes, the pupil is offset to the left from the center of the field of view, and the top of the cornea is offset to the left from the middle a-line of the central B-scan. The sample arm shaded in blue corresponds to a position where the lateral origins of the TRF and ERF coincide. The position of the eye and corresponding central B-scan are found below in the blue boxes. In the blue boxes, the pupil is in the center of the field of view and the top of the cornea is in the center of the central B-scan.

Step 4: Determine the origin of the ERF along the Z-axis. This was accomplished by setting the origin of the ERF 1.5 mm underneath the apex of the cornea along the Z-axis of the ERF (found in the previous step).

Repeat steps 2 and 3 if 1) the z-axis of the TRF and Z-axis of the ERF are not parallel or 2) the x and y origin of the TRF are not well aligned with the apex of the cornea.

Step 5: The remaining DoF of RPW angle (orientation of X, Y-axis within the defined XY-plane in ERF) were determined by the given mounting position of the robot and mouse holder on the same optical table.

After the alignment of the z-axis of the TRF and the Z-axis of the ERF, the XY-axes of the ERF are oriented in the same direction as the current xy-axes of the TRF after step 3. Before each imaging session, the default position of the TRF was set to a default pose relative to the robot base, where the orientation of the xy-axes of the TRF relative to the direction of the edges of the optical table the robot is mounted on is known. The xy-axes of the TRF undergo rotations during steps 2 and 3.

Step 6: Map out trajectory for rotating the TRF around the ERF. Following the definition of ERF, the WRF was set as the ERF (simply a nomenclature change). Next, conformal scanning of the outflow pathway was performed to acquire data. The TRF was rotated 60 degrees around the X-axis of the ERF. Empirically, the outflow pathway is approximately 60 degrees from the center of the ERF so this makes the scanning conformal to the outflow pathway. The TRF was rotated 360 degrees around the Z-axis of the ERF with minor adjustments in position made to optimize signal quality. During minor adjustments, the top surface of the first and last B-scan in the TRF was adjusted to be the same height along the fast axis. Additionally, SC was moved into the central A-line in the central B-scan and to a fixed reference arm position. Additional rotations can be made around the TRF xy-axes if the user is unsatisfied with how conformal the scan is to the surface although this usually is not necessary. For this the minor axis of an ellipse fit to SC should orient along the z-axis of the TRF. This was done by moving the TRF, effectively optimizing the TRF position.

If the user was unsatisfied with the ERF origin positioning, in other words, if there is a major adjustment needed in each scanning position, the user can do a quick preview scan while the Z-axis is rotated about the ERF. At 4 calibration positions during the preview scan, the position of the TRF can be manually optimized for a better signal quality of the OCT image of the cornea top surface and the appearance of a leveled cornea top surface in OCT B-scan, and then the robot joint angles are recorded. The robot joint angles at these 4 positions as well as those during the original alignment position were input to the “Define TCP” tool of a commercial robot motion simulation software (RoboDK) to refine the origin of the ERF.

Following the alignment of the lateral origins of the ERF and TRF, the direction of the z-axis of the ERF was identified (FIG. 4E). The blue and red shaded sample arms in the top panel correspond to positions where the z-axis of the TRF is parallel and oblique to the z-axis of the ERF respectively. The images below in the blue and red shaded boxes correspond to the orientation of the eye as viewed from the blue and red shaded sample arms and corresponding B-scans respectively. In the shaded red boxes, a line drawn through the left and right iris is tilted downwards to the right. The iris is also tilted downwards to the right in the corresponding central B-scan. In the shaded blue boxes, a line drawn through the left and right iris is even on both sides of the lens. The iris axial position was observed to be even on the left and right sides of the lens in the corresponding central B-scan.

After determining estimates for the lateral origin and z-axis of the ERF, the origin of the ERF was refined using a calibration process (FIG. 4F). Following identification of the eye's optical axis, the sample arm is rotated 60 degrees about the y-axis of the estimated ERF. At this point, the limbal region of the eye is within the OCT image plane. However, the identified ERF may not be perfect and adjustments are often needed. The blue box shows a case where Schlemm's Canal (SC) is in the center of the image and the minor axis of SC is roughly parallel to the sample arm beam. The red box shows a case where SC is not centered and the minor axis of SC is oblique to the sample arm beam. Overall, the calibration step involves making minor adjustments to the robotic arm position to center the SC within the OCT central B-scan and align the z-axis of the TRF roughly parallel to the z-axis of the minor axis of SC. The calibration process involves recording the robotic joint position for 3 orientations used to image the outflow pathway. These positions are input into the “Define TCP” tool of RoboDK, whose calculated TCP is set as the true origin of the ERF. Following the measurement of the ERF origin and z-axis orientation, the WRF was set to have the same origin and z-axis orientation. Afterward, the TRF was rotated around the z-axis of the WRF and acquire 8 separate OCT volumes separated by 45 degrees each (FIG. 4G).

### Real-Time Corneal Apex Segmentation

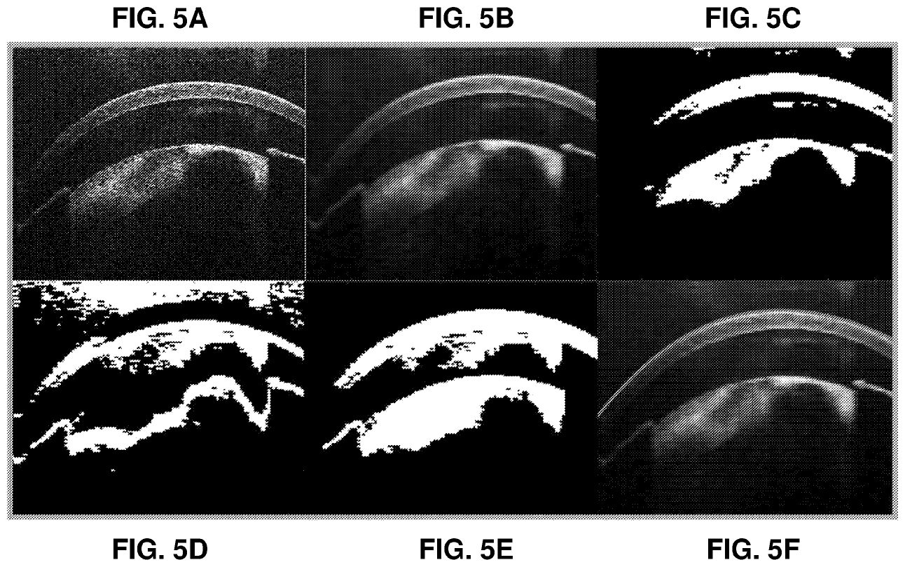

Relative positions of the lateral origin of the TRF and the ERF were determined using real-time segmentation of the cornea. After bringing the central cornea into the field of view of the OCT image plane, real-time image processing of the central B-scans were used to identify the relative lateral positions of the TRF with respect to the ERF. An initial raw image was acquired in real-time (FIG. 5A) and mean-filtered (FIG. 5B) to increase signal quality. The original image was sized 1024×128 and the mean-filter had size 9×3. For faster processing, the image was downsampled by a factor of 8 in the axial direction, at which point the preview image was sized 128×128. The cornea and lens were automatically segmented using a modified hysteresis thresholding scheme when an unbalanced preview was used and thresholding when a balanced preview was used. Hysteresis thresholding steps are useful for getting rid of auto-correlation near the zero-delay line, as the method works for both balanced and unbalanced preview. The image was binarized with the application of an automatically defined high threshold (85th percentile of image intensity, FIG. 5C), with regions less than 20 percent of the largest connected binarized region removed. Next, the image was binarized with a low threshold (65th percentile, FIG. 5D), with regions binarized by the high threshold removed. Components connected to the borders were removed, eliminating any autocorrelation component remaining after thresholding, and the subsequent images binarized with the low and high thresholds were merged to form a structural image mask (FIG. 5E). A third-degree polynomial was fit to the top of the binarized image (FIG. 5F) to identify the top surface of the cornea. The lateral position of the apex of the fitted cornea were used to estimate the origin of the ERF along the scanning direction.

The relative orientation of the z-axis of the TRF and ERF along the fast scanning axis were evaluated using the slope of the center of mass in the axial direction as a function of transverse position x. The center of mass was calculated as a function of position x with the following equation, where I(z, x) is the intensity of the central B-scan at depth z and position x:

$${COM}_{y} ( x ) = \frac{{\sum }_{z} I ( z , x ) z}{{\sum }_{z} I ( z , x )}$$

After COM_{y}(x) was calculated, a line was fit to COM_{y}(x) and the fitted slope was used as an estimate for relative alignment of the z-axes of the TRF and ERF. When the z-axes of the TRF and ERF are well aligned, the fitted slope has a value near zero. The greater the misalignment between the z-axes, the higher the absolute value of the fitted slope, with the sign of the slope giving an indication of whether the z-axis of the TRF was aligned clockwise or counterclockwise to the z-axis of the ERF along the fast scan axis.

After the central cornea was brought into the OCT field of view, B-scans previewing the central cornea along both the fast and slow axes were analyzed in real time to align the transverse origin and z-axis direction of the TRF and ERF using the methods described supra.

The cornea was segmented and the position of the transverse origin of the ERF was estimated with the top of the segmented cornea. Defining T_{o,x} as x position of the TCP and E_{o,x} as the location of the top of the cornea in x-axis, the TRF was translated by α(T_{o,x}−E_{o,x}) in the x direction. This process was repeated until the top of the cornea aligned with the transverse position of the TCP.

The misalignment of the z-axes of the TRF and ERF was estimated along the direction of the scanning axis with the y-center of mass slope along that axis. Defining Y_{tilt,x} as the y-center of mass slope for the B-scan corresponding to the x-axis, the TRF was rotated by angle βY_{tilt,x} around the y-axis of the TRF. The same steps were done for the B-scan corresponding to the y-axis of the TRF with the difference being that the TRF was rotated around the x-axis of the TRF. Following the alignment of the TRF lateral origin and z-axis orientation relative to the ERF, the lateral origin and the z-axis direction of the WRF were set to align with the TRF.

Following the initial alignment of the transverse origin and the z-axis of the WRF and ERF, the alignment of the origin of the WRF was refined. For a first estimate, the origin of the WRF was set to be 0.9 mm below the TCP in the z direction of the TCP. The current joint pose of the robotic arm used to image the central cornea was recorded.

After the initial estimate of the WRF, corresponding to the ERF, the TRF was rotated by 60 degrees around the y-axis of the WRF. Following the rotation of the TRF, the position and orientation of the TRF was adjusted until SC was in the center of the OCT field of view and the minor axis of SC aligned with the z-axis direction of the TRF. At this point, the posture of the robotic arm was recorded. The TRF was rotated by 120° around the z-axis of the WRF, the position of the TRF was adjusted, and the posture of the robotic arm was recorded. The last step was repeated once more. The recorded postures were input into the “Define TCP” tool of the RoboDK software and the origin of the WRF was set as the newly defined point by the RoboDK software, with the idea being that the origin of the WRF ought to align with the centroid of the outflow pathway positions recorded in the pre-calibration process.

Influence of vis-OCT Illumination Incident Angle

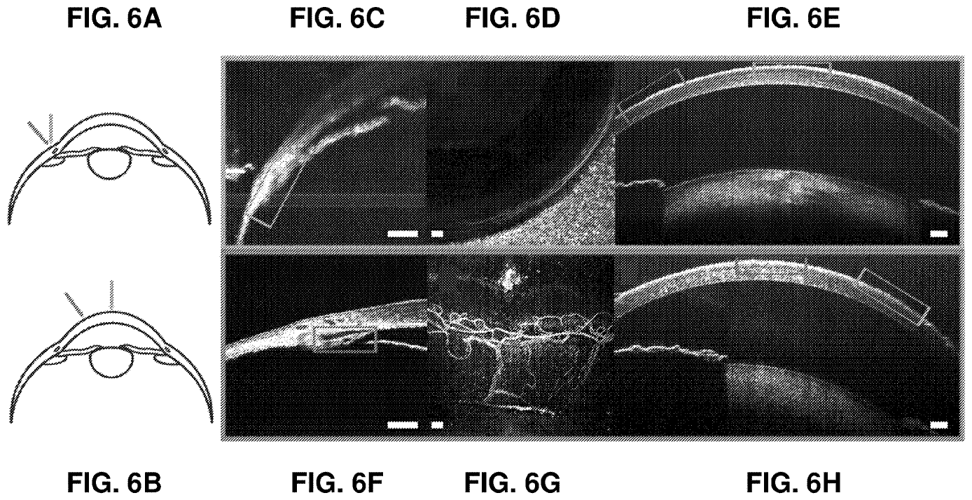

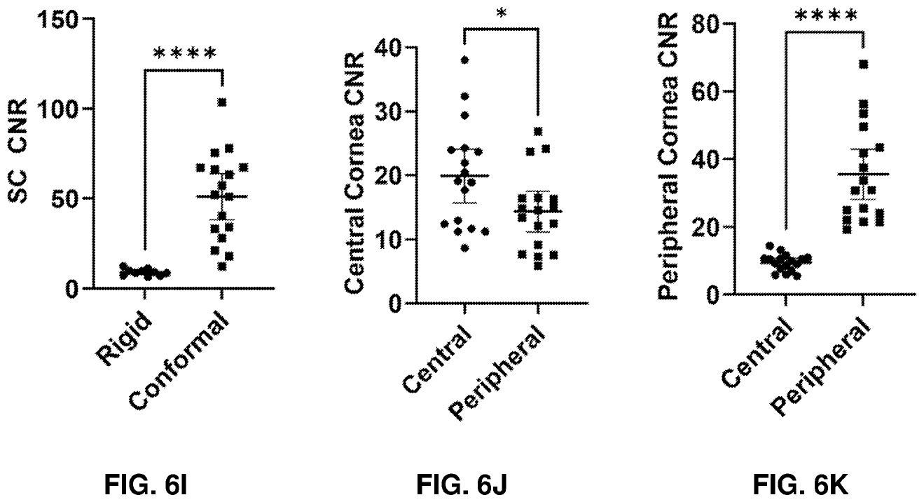

The incident angle of the OCT beam influences the contrast-to-noise ratio (CNR) of the cornea and outflow pathway. Since the axial resolution of OCT is several folds better than lateral resolution, anatomical features with dimensions smaller than the lateral resolution of OCT are better resolved when the illumination beam is parallel to the feature. A key advantage of freeform OCT is its capability of adjusting incident angle as needed. If each cross-sectional region of SC is conceptualized as an ellipse, SC's minor axis corresponds to its smaller dimension and major axis to its larger dimension. Both the SC and the cornea were imaged with the incident beam normal to the major axis of SC (FIG. 6A) and the central cornea (FIG. 6B).

When the vis-OCT illumination aligns with the normal axis of the cornea, it was difficult to resolve the borders of SC (FIG. 6C) and hard to visualize the limbal vasculature from the OCTA (FIG. 6D). Different corneal layers in the central cornea were differentiated but not the peripheral cornea (FIG. 6E). When the vis-OCT illumination aligns with the minor axis of SC, the borders of SC (FIG. 6F) and the limbal vasculature network (FIG. 6G) were resolved. Additionally, different corneal layers in the peripheral cornea but not the central cornea (FIG. 6H) were observed. Consistent with qualitative observations, the CNR between SC and surrounding tissue was 9.0±1.8 when the OCT beam was orientated normal to the central cornea and 51.0±24.7 when orientated along the minor axis of SC (FIG. 6I). The CNR between the epithelium and stroma along the central cornea was 19.9±8.2 when orientated normal to the central cornea and 14.3±6.1 when orientated along the minor axis of SC (FIG. 6J). The CNR between the epithelium and stroma along the peripheral cornea was 9.5±2.5 when orientated normal to the central cornea and 35.5±14.4 when orientated along the minor axis of SC (FIG. 6K). Accordingly, a vis-OCT illumination beam normal to the sample surface, which is achieved by freeform OCT conformal scanning, provides superior image quality.

### Reconstruction of Civ of the Anterior Segment

To reconstruct a CIV of the anterior segment, eight vis-OCT volumes, separated by 45 degrees along the trajectory illustrated in FIG. 2B, were acquired. The OCT volumes (i.e. volumetric images) were acquired with a visible light robotic AS-OCT system having a wavelength range of 510 nm-610 nm. The incident power on the eye was 1 mW and the speed of data acquisition was 75 kHz. A field of view of 2.04 mm was used for the lateral directions. A temporal speckle averaging data acquisition pattern was used to acquire each volume, where each b-scan was acquired twice per subvolume and three sub-volumes were acquired. B scans from each sub-volume were subsequently averaged.

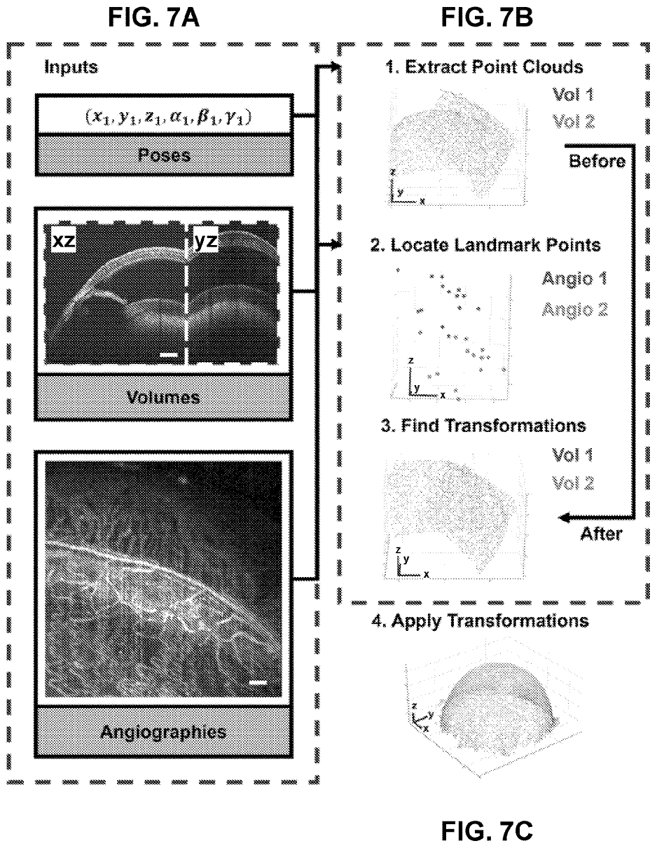

For each volume, the orientation of the TRF with respect to the ERF was recorded and both the microanatomy and angiogram were reconstructed (FIG. 7). Processing of structural OCT images was done according to prior methods, and angiography data was processed using both the OMAG and SSADA algorithms (see Jia, Y. et al. Split-spectrum amplitude-decorrelation angiography with optical coherence tomography. Opt Express 20, 4710-4725 (2012); see also Yi, J., Chen, S., Backman, V. & Zhang, H. F. In vivo functional microangiography by visible-light optical coherence tomography. Biomed Opt Express 5, 3603-3612 (2014)).

After overlapping volumes were determined based on the recorded TRF orientations, adjacent volumes were montaged. To montage adjacent volumes, the tissue surface was segmented and the outer surface was represented as a point cloud (see Li, L., Wang, R. & Zhang, X. A Tutorial Review on Point Cloud Registrations: Principle, Classification, Comparison, and Technology Challenges. Mathematical Problems in Engineering 2021, U.S. Pat. No. 9,953,910 (2021)) (step 1 in FIG. 7). Common vessel branch points in adjacent volumes were identified, and the outer surface at the lateral position of the branch points were extracted (step 2 in FIG. 7). Afterwards, the M-estimator Sample Consensus algorithm (see Torr, P. H. S. & Zisserman, A. MLESAC: A New Robust Estimator with Application to Estimating Image Geometry. Computer Vision and Image Understanding 78, 138-156 (2000)) was used to determine rigid geometric transformation between the common branch points (step 3 in FIG. 7). After determining the rigid geometric transformation between overlapping regions of adjacent point clouds, the mean distance between each point on a point cloud and the closest point on the adjacent point cloud was determined to be 9.6±2.3 μm. All point clouds were transformed to the ERF coordinate system (i.e., the WRF). Following transformation into the ERF, overlapping regions of the point clouds were identified (FIG. 7) and the iterative closest point algorithm (see Besl, P. J. & McKay, N. D. A method for registration of 3-D shapes. IEEE Transactions on Pattern Analysis and Machine Intelligence 14, 239-256 (1992)) was used to refine the transformations of each reference frame relative to the ERF. Additional details of the process used to reconstruct the compound volumetric image of the CIV of the anterior segment are provided infra.

### Simultaneous Localization and Mapping for OCT Volumetric Montaging

To montage the volumetric OCT data acquired from different poses, simultaneous localization and mapping (SLAM) was implemented to merge the OCT volumes. SLAM is the computational process of mapping out an unknown environment in robotics. Typically, a combination of a robot's measured position within an environment and sensor readings are used to localize the position of the robot within the environment. Since the accuracy of the robot's measured position is limited, various algorithms exist to correct the measured position using external sensors on the robot. For reconstructing the CIV of the anterior segment, the location of the robot relative to the eye was measured by recording the orientation of the TRF relative to the WRF for each volume. However, errors in the accuracy of the robot position necessitate refinement of the measured position. To correct positional errors, anatomical features in the overlapping regions between volumes were matched. In other words, the acquired volumes served as the external sensors used to correct errors in the position of the robot.

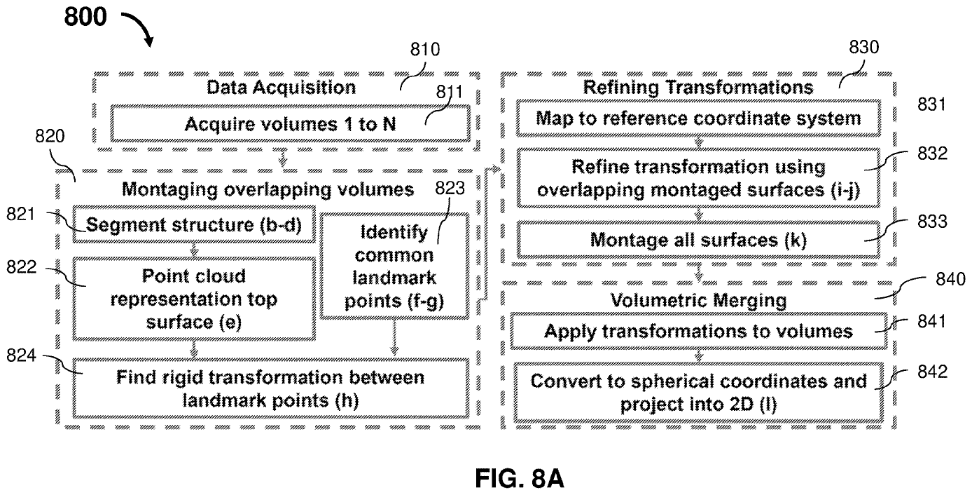

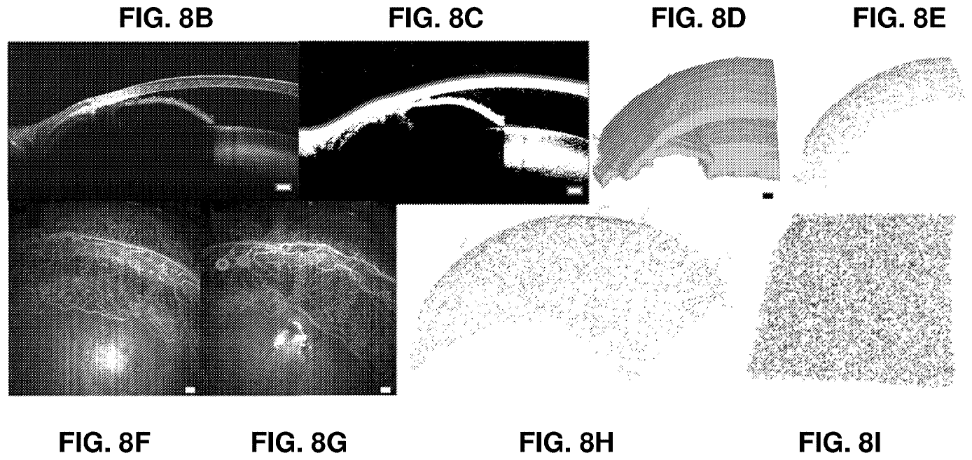

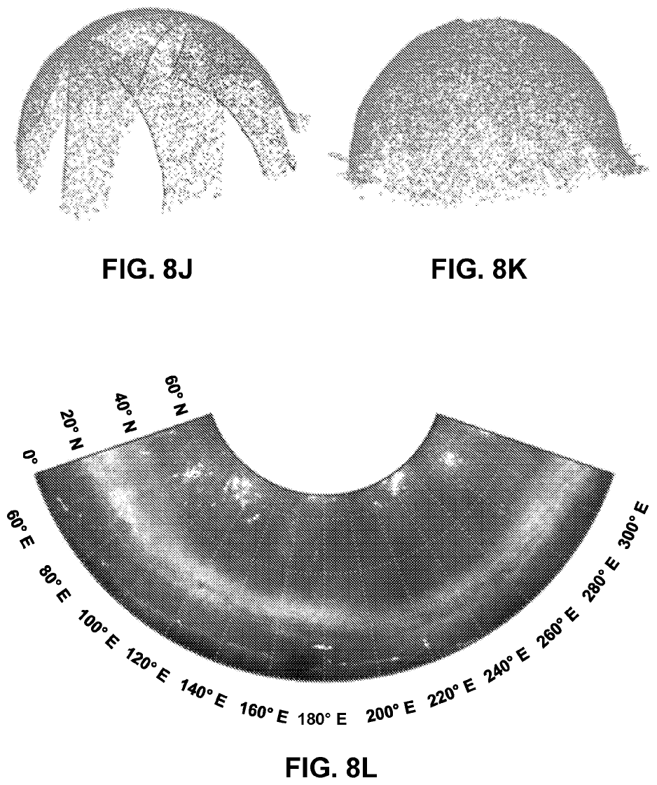

FIG. 8A is a flow diagram illustrating a SLAM process 800 for OCT volumetric reconstruction and montaging. At 810, the SLAM process 800 carries out data acquisition. Data acquisition 810 involves acquiring a plurality of individual OCT volumetric images at 811.

At 820, the SLAM process 800 performs montaging of overlapping volumes. The montaging of overlapping volumes includes segmentation of the structure being imaged at 821, providing a point cloud representation of the top surfaces of the volumes imaged in the plurality individual OCT volumetric images at 822, identifying common landmark points in pairs of the plurality of individual OCT volumetric images at 823, and finding rigid transformations between landmark points at 824.

Segmentation of the eye surface and representation of top surfaces as point clouds is pictured in FIGS. 8B through 8H. For each volume, the top surface of the eye closest to the incident OCT beam was represented as a point cloud. I(x, y, z) was defined as the intensity at depth z and lateral position x of the y^{th} B-scan (FIG. 8B). Every B-scan was thresholded and binarized and the largest spatially connected remaining object was retained (FIG. 8C). Each binarized B-scan was merged to form a binarized volume. After segmentation, a continuous corneal and limbal structure (FIG. 8D) was generated. For every A-line in the volume, the axial position z of the top surface of the spatially connected binarized set of points was recorded and the set of points was represented as a point cloud (FIG. 8E). To determine whether two volumes A and B overlap, an assessment of whether the spatial domain of A and B in the WRF have greater than 20% volumetric overlap. To determine the spatial domain of volume A in the WRF, the relative pose of the TRF is applied to the WRF as recorded by the robotic arm to the spatial domain of A in its own image reference frame, which is a fixed domain based on OCT imaging depth and galvanometer scan range. If A and B overlap, the two point clouds of A and B are merged with the following procedure.

Let P_{A} be the point cloud for the top surface of volume A. Furthermore, let S_{A} and S_{B} be the functions returning the axial position of the eye surface for two overlapping volumes A and B. For an arbitrary surface N, if the top surface of the segmented surface at (xᵢ, yᵢ) is zᵢⱼ, then:

$${S}_{N} ( {x}_{i} , {y}_{j} ) = {z}_{i j}$$

The lateral positions of 15 different common landmark points, usually vessel branch points in the OCTA, are identified in the overlapping region between A and B (FIGS. 8F-8G). Supposing that the landmark points have lateral positions {(x₁, y₁), . . . , (x₁₅, y₁₅)} in volume A, then the set of landmark points L_{A} can be defined as:

$${L}_{A} = \{ ( {x}_{1} , {y}_{1} , {S}_{A} ( {x}_{1} , {y}_{1} ) ) , … , ( {x}_{15} , {y}_{15} , {S}_{A} ( {x}_{15} , {y}_{15} ) ) \}$$

The landmark points L_{B} in volume B are defined accordingly. The M-estimator sample consensus algorithm (see Torr, P. H. S. & Zisserman, A. MLESAC: A New Robust Estimator with Application to Estimating Image Geometry. Computer Vision and Image Understanding 78, 138-156, doi:https://doi.org/10.1006/cviu.1999.0832 (2000)) was subsequently used to determine the rigid transformation T_{AB} mapping L_{A} onto L_{B} (FIG. 8H).

At 830, the SLAM process 800 refines the rigid transformations. After the identification of all rigid transformations, all the point clouds are mapped onto the coordinate system of the WRF at 831. To do this, the rigid transformation mapping P_{A} to the WRF is defined by the relative pose of the TRF to WRF when volume A was acquired. Since rigid transformations are linear operators, the rigid transformation between volumes can be applied in sequence to map all volumes onto the same coordinate system. If T_{AWRF} maps P_{A} to the WRF coordinate system, then T_{BA}T_{AWRF} would map P_{B} to the WRF coordinate system. When a series of transformations are applied consecutively, errors from each transformation can cascade. When montaging individual OCT volumetric images to construct a compound volumetric image, this may result in a mismatch between surfaces if a volume is overlapping with multiple different volumes. Accordingly, the process of refining the rigid transformations includes refining the transformations using overlapping montaged surfaces at 832 and montaging all surfaces at 833.

Refining of the rigid transformations is illustrated in FIGS. 8I through 8K. For each pair of point clouds P_{A} and PB that were montaged together, the points corresponding to the overlapping regions between P_{A} and P_{B} were determined (FIG. 8I). After applying T_{AB} to P_{A}, P_{A} and P_{B} reside in the same coordinate system. For each point in P_{A}, the closest point in P_{B} was found. The union of closest points in P_{B} to P_{A} was considered the overlapping region in P_{B}, which was denoted O_{BA}. The overlapping region O_{AB} was determined accordingly. Oₐₗₗ was defined as the union of all overlapping regions between every overlapping volume. After mapping all sets of overlapping regions onto the WRF, the transformation mapping each volume to the WRF was refined. To refine the transformation mapping volume B to the WRF, the set of overlapping regions O_{BA} and O_{BC} was determined. The iterative closest point algorithm (see Li, L., Wang, R. & Zhang, X. A Tutorial Review on Point Cloud Registrations: Principle, Classification, Comparison, and Technology Challenges. Mathematical Problems in Engineering 2021, 9953910, doi:10.1155/2021/9953910 (2021)) was used to fine-tune the transformation mapping O_{BA} and O_{BC} onto Oₐₗₗ−(O_{BA} ∩O_{BC}). This process is applied to every volume consecutively to refine the transformation of all volumes to the WRF (FIG. 8J). After refining every transformation a first time, the process of refining every transformation was repeated two additional times. Following transformation refinement, the outer surface for all point clouds mapped onto the reference world coordinate system was smooth and continuous (FIG. 8K).

At 840, the SLAM process 800 performs volumetric merging of the imaging data of the acquired volumetric images. The volumetric merging at 840 includes applying transformation to the acquired volumetric images at 841. After identifying the rigid transformations T_{A} mapping P_{A} from its original image reference frame to a common global world reference frame for every volume, each transformation is applied to each volume. If T_{A} maps point (x,y,z) from the original image coordinate system A to point (i,j,k) in the reference world coordinate system, then:

$${I}_{w o r l d} ( i , j , k ) = {I}_{A} ( x , y , z ) .$$

When multiple points from different volumes are mapped onto the same point in the world coordinate system, the maximum intensity of the volumes mapping onto that point can be taken. In other words, if T_{A} maps (xₐ, yₐ, zₐ) onto (i,j,k) and T_{B} maps (x_{b}, y_{b}, z_{b}) onto (i,j,k), then I_{world}(i, j, k) would equate to the maximum of I_{A}(xₐ, yₐ, zₐ) and I_{B}(x_{b}, y_{b}, z_{b}).

The volumetric merging at 840 includes converting the representation of the imaging data into spherical coordinates at 842. The reconstructed 3D volume is transformed from cartesian to spherical coordinates using the following transformation:

$${l}_{s p h e r i c a l} ( r , θ , ϕ ) = {I}_{world} ( r sin θcosϕ , r sin θsinϕ , r cos θ )$$

En-face projections E were created as a function of θ and φ:

$$E ( θ , ϕ ) = \frac{{\sum }_{r = {r}_{-}}^{r = {r}^{+}} {I}_{s perical} ( r , θ , ϕ )}{{\sum }_{r = {r}_{-}}^{r = {r}^{+}} 1}$$

As a spherical surface cannot be perfectly projected onto a plane, the various projections from cartography (see Lapaine, M. & Frančula, N. Map Projections Classification. Geographies 2, 274-285 (2022)) were applied to E(θ, φ) to render the eye in 2D (FIG. 8L).

### Reconstruction of the Digital Twins of the Anterior Segment

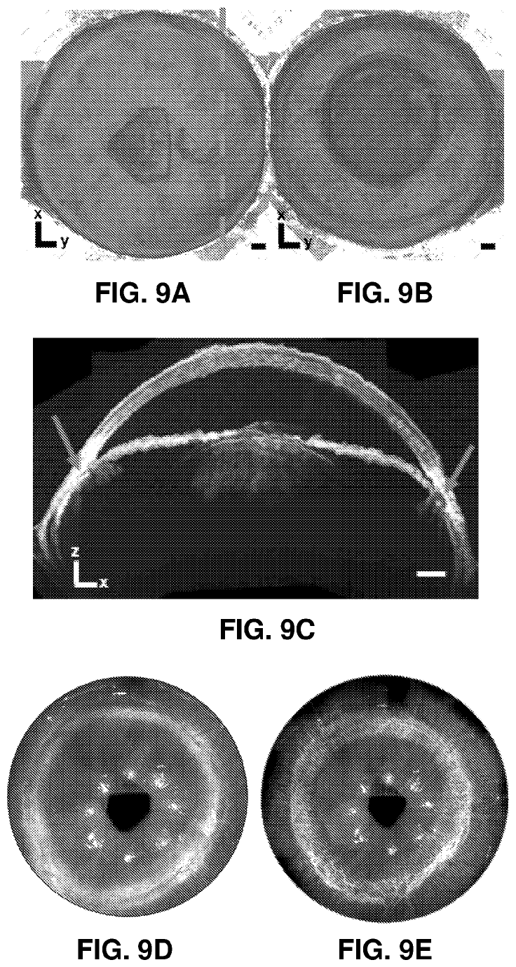

The calculated geometric transformations were applied to each imaging volume and the anterior segment was reconstructed from the anterior and posterior perspectives (FIGS. 9A-9B). A sample cross-section located 620 μm from the center of the eye shows a smooth corneal and iris surface, indicating accurate montaging (FIG. 9C). The coordinate system of the reconstructed volume was converted from a cartesian coordinate into a spherical coordinate system, and the mean structural and angiographic signal was found as a function of θ and φ. Following the change of coordinate system, the structural and angiographic volumetric data was projected into 2D using a stereographic projection (see Kosel, T. H. Computational techniques for stereographic projection. Journal of Materials Science 19, 4106-4118 (1984)) (FIGS. 9D-9E). Features, including the trajectory of individual blood vessels, along the projections are smooth and continuous between montaged volumes.

### Segmental Pattern of Sc Morphology

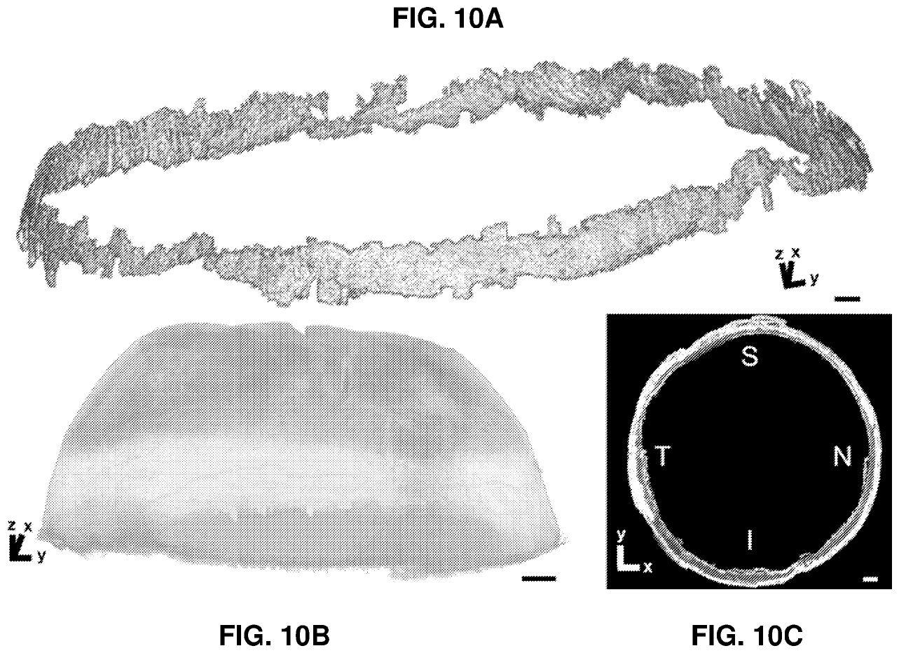

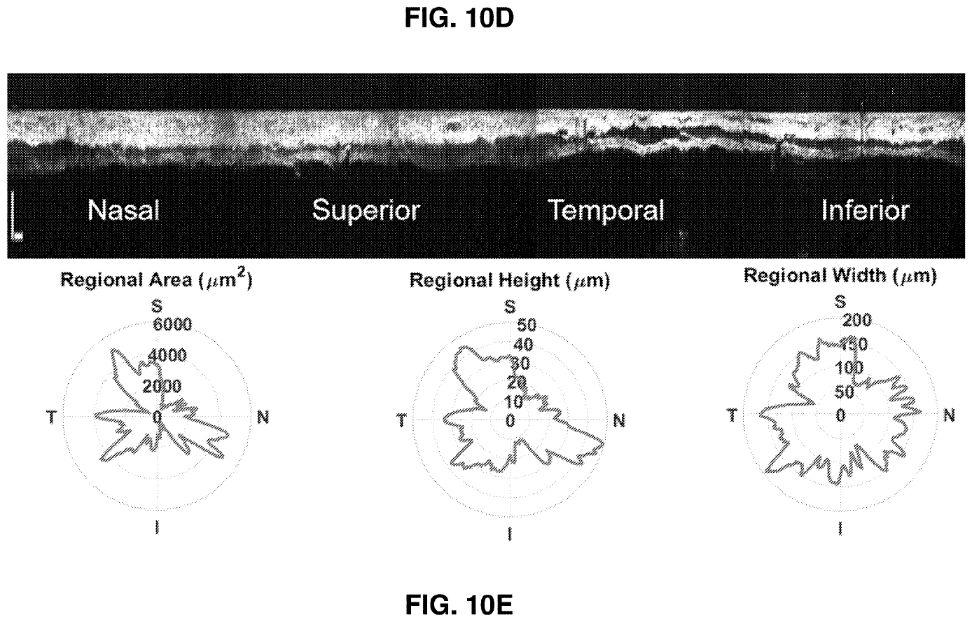

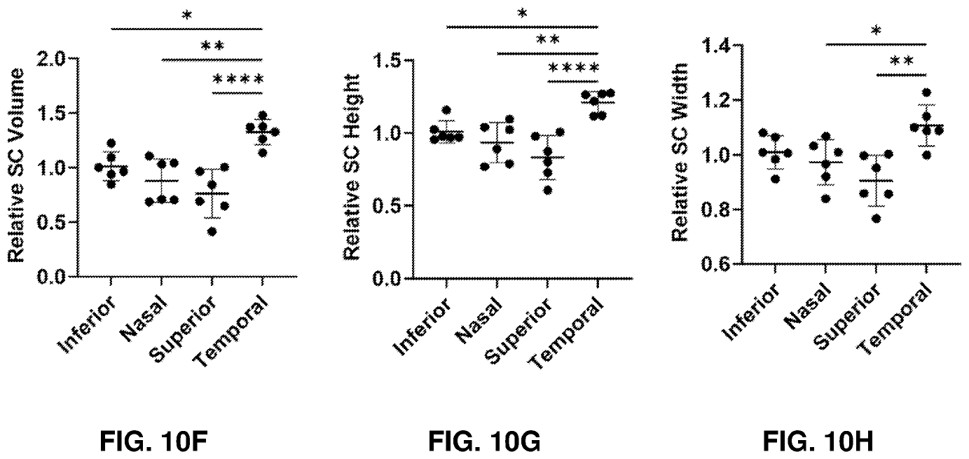

From the digital twin of the anterior segment, the SC around the entire globe was reconstructed (FIG. 10A). Further details of the reconstruction of the SC are provided infra. As expected, SC forms a continuous lumen around the periphery of the eye (FIG. 10B). Each cross-section of SC has a roughly ellipsoidal shape, with the major axis corresponding to the width of SC and minor axis to height. When visualized along the width dimension, SC forms a continuous hyporeflective ring immediately lateral to the anterior chamber (FIG. 10C). Segmental patterns in SC height were observed, with SC height being larger in the nasal and temporal quadrant for a C57BL/6 mouse (FIG. 10D). For each mouse, segmental patterns in SC area, height, and width were measured as a function of angle around the globe (FIG. 10E).

Relative to the mean volume across all quadrants for each specific eye, SC volume was 32.6±11.7% larger in the temporal quadrant, 23.8±22.2% smaller in the superior quadrant, 12.0±19.9% smaller in the nasal, and 1.2±13.1% larger in the inferior quadrant. Volume in the temporal quadrant was greater as compared to the other quadrants (FIG. 10F). Relative to the mean height across all quadrants, SC height was 21.0±7.4% larger in the temporal quadrant, 16.7±15.2% smaller in the superior quadrant, 6.6±13.8% smaller in the nasal, and 0.9±7.7% larger in the inferior quadrant. Height in the temporal quadrant was greater as compared to the other quadrants (FIG. 10G). Relative to the mean width across all quadrants, SC width was 10.7±7.5% larger in the temporal quadrant, 9.5±9.3% smaller in the superior quadrant, 2.8±8.3% smaller in the nasal, and 0.9±6.1% larger in the inferior quadrant. Width in the temporal quadrant was greater as compared to the nasal and superior quadrants (FIG. 10H).

### Segmental Distribution of CCS

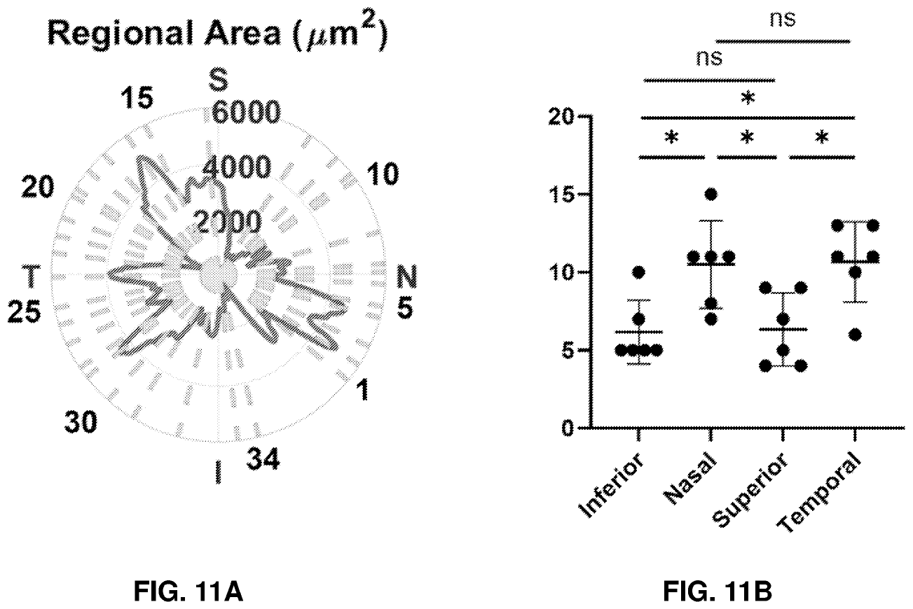

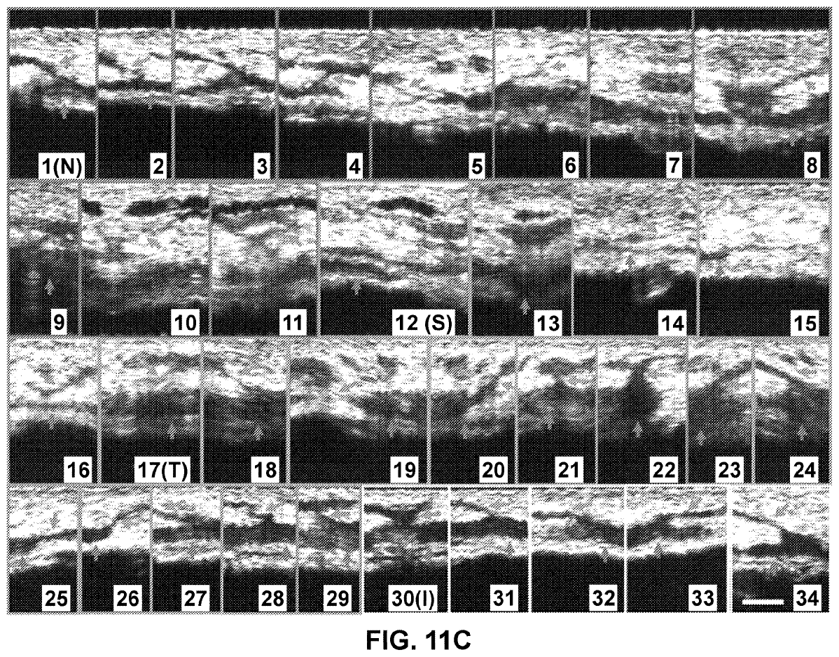

As the segmental distribution of CCs has been hypothesized to influence MIGS outcomes, each CC was visualized around the globe. Positions of CCs around the eye were mapped and locations were correlated with the local SC area in C57BL/6 mice (FIG. 11A). The mean number of CCs for mice was found to be 33.7±6.5. The average number of CC in the inferior, nasal, superior, and temporal quadrants are 6.2±2.0, 10.5±2.8, 6.3±2.3, and 10.7±2.6 respectively (FIG. 11B). Overall, the nasal and temporal quadrants had more CCs than the inferior and superior quadrants (p<0.05).

The OCT image was resampled, as described infra in connection with the reconstruction of SC, to view each CC in a C56BL/6 mouse (FIG. 11C). For each resampled image, the connection between SC (blue arrows) and CC (green arrows) was illustrated. Several different morphologies were observed, with some channels open throughout, some exhibiting pinch points, some exiting at a vertical angle, and some exiting at a side angle. Some examples are illustrated with channel 30 open throughout, channel 6 having a pinch point, channel 22 exiting at a vertical angle, and channel 33 exiting at a side angle.

### Eye Exposure

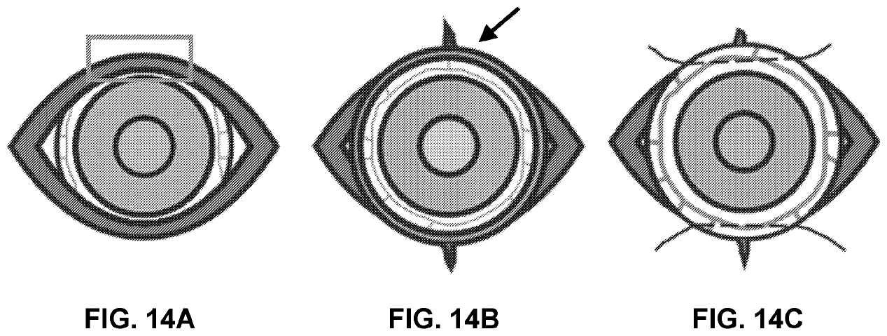

As the eyelids typically block the nasal and temporal limbal region (FIG. 14A), these areas were exposed to image the entire limbal region around 360 degrees of the eye. First, a small cut was made to the nasal and temporal portions of the eyelid and a speculum was inserted underneath the eyelids (FIG. 14B). An alternative to exposing the eyelid is the propose the eye and suture the eyelids together underneath the eye (FIG. 14C). However, this process causes blood reflux, which significantly enlarges the conventional outflow pathway.

### Statistical Analysis

All statistical comparisons unless otherwise specified were done after performing a one-way ANOVA using GraphPadPrism 9.5.1. When multiple comparisons were made, a Tukey multiple comparison correction was performed. The F-test was used to determine whether the fit slopes of any fit line were significantly different from zero.

### Reconstruction of Schlemm's Canal

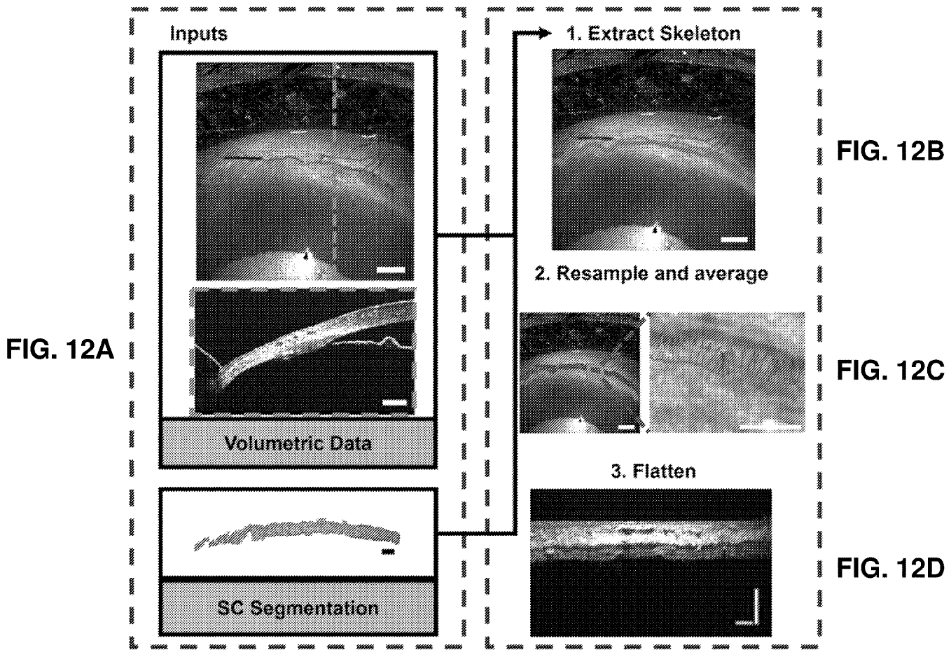

To reconstruct the entire SC, the SC was segmented for every B-scan (FIG. 12). First, a point contained within SC was manually selected every 30 B-scans. For each of these B-scans, a flood-fill technique was used to determine the borders of SC. For this technique, the set of continuous pixels with grayscale intensity below the intensity of the chosen point plus a tolerance value was found. Following the semi-automated segmentation of SC every 30 B-scans, the centroid of the SC in each of those B-scans was determined. Based on the SC centroid position, the SC centroid position was interpolated for the scans that had not yet been segmented. Next, the same flood-fill technique as before was used to determine the borders of SC for all B-scans. Manual correction to the segmentation was applied when necessary. All CCs were manually segmented.

In order to reconstruct SC along its height dimension, resampling was performed. Defining G as the 3D volumetric segmentation of SC, the binarized projection mask of SC within each individual volume is the set of points (x,y) satisfying:

$${\sum }_{z} G ( x , y , z ) > 0 .$$

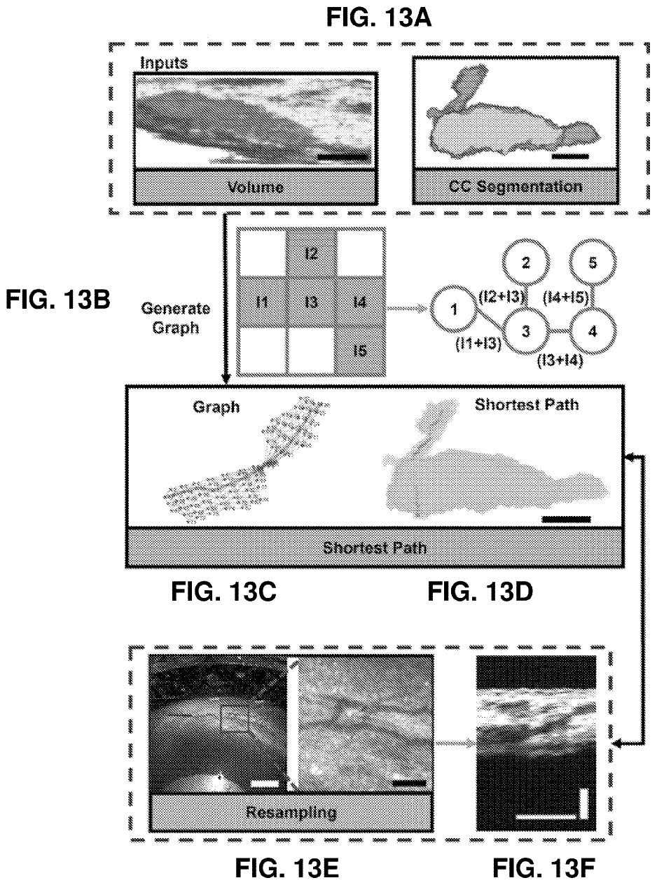

Overall, the binarized projection mask forms a continuous structure, reflecting the connectivity of SC (blue overlay in FIG. 13). The binarized mask of SC was skeletonized and all branches except for the largest one were pruned. Based on the coordinates of the skeleton, SC was resampled along the skeleton, with lateral averaging occurring along the normal direction of the skeleton to improve SNR. Specifically, the intensity along the skeleton centerline and 5 μm along the normal direction of the skeleton were averaged together (FIG. 13). Each Letting X and Y be the ordered set of points along the skeleton, the normal direction is defined as:

$$∇ ( skeleton ) = ( - \partial Y , \partial X )$$

In this case, ∂Y and ∂X were numerically calculated by taking the central difference of X and Y. Each A-line of the resampled image corresponds to the A-line at a lateral position of the skeleton in the original volume. Following the resampling of SC, the top surface of the eye was flattened to be along the same depth position in the resampled image (FIG. 13). Flattening was done by thresholding the resampled image, taking the largest continuous region, and translating each A-line such that the top border of the segmented image ended up at the same vertical depth position. To stitch the resampled SC between overlapping volumes, the positions where SC overlapped in the global WRF coordinate system were determined after SLAM. The skeleton points in the overlapping region were determined and matched for both overlapping volumes. For each matched skeleton point, resampling was performed at the position closer to the center of the original single-volume field of view.

For the set of points in the binarized mask of SC, the mean depth of SC along each position z_{sc}(x,y) was found.

$${z}_{s c} ( x , y ) = \frac{{\sum }_{z} G ( x , y , z ) z}{{\sum }_{z} G ( x , y , z )}$$

The intensity of the resampled projected image at P is:

$$P ( x , y ) = {I}_{world} ( x , y , {z}_{SC} ( x , y ) )$$

For lateral positions not contained in the binarized mask of SC, z_{SC}(x, y) was interpolated at these points using a nearest neighbor approach to find the depth to resample at.

Graph theory was utilized for the reconstruction of collector canals. Given a segmentation of each CC and volumetric data, each CC connecting SC to the distal vasculature was projected. Each CC was manually segmented. Defining C as a set of voxels in the 3D segmentation for an individual CC and the adjacent SC to the CC (FIG. 13), C can be modeled as a graph, where every voxel in C is represented as a node and spatially connected voxels have an edge between them (FIG. 13). If the points (xᵢ, yᵢ, zᵢ) and (xⱼ, yⱼ, zⱼ) were spatially connected in C, the weight of the edge between the nodes associated with points (xᵢ, yᵢ, zᵢ) and (xⱼ, yⱼ, zⱼ) is:

$$W ( ( {x}_{i} , {y}_{i} , {z}_{i} ) , ( {x}_{j} , {y}_{i} , {z}_{j} ) ) = I ( {x}_{i} , {y}_{i} , {z}_{i} ) + I ( {x}_{j} , {y}_{j} , {z}_{j} )$$

The two end nodes of the graph were determined by finding the point in C with the lowest depth position relative to the eye surface in the first and last B-scan containing any voxel of C. The same process was repeated for the point with the highest depth position. In the case that the first B-scan had the point with the highest depth dimension, the end nodes corresponded to the highest voxel in the first B-scan and the lowest voxel in the last B-scan. Otherwise, the end nodes consisted of the lowest voxel in the first B-scan and the highest voxel in the last B-scan. Following the identification of end nodes, the shortest path in the graph between the end nodes (FIG. 13) was determined. The nodes contained within the shortest path were converted to physical voxels within C (FIG. 13). The lateral position for each of the voxels was taken, with repeated positions removed, as points to resample for the projection of the CC (FIG. 13). No averaging along the normal direction was done as averaging obscures visualization of CC at its smallest point. After resampling along the lateral positions corresponding to the voxels in the shortest path, a clear lumen connecting SC to the distal vasculature is easily visualized as the resampled CC. (FIG. 13).

### Real-Time Image Processing for Alternative Methods to Identify the Lateral Position and Orientation of the Central Cornea

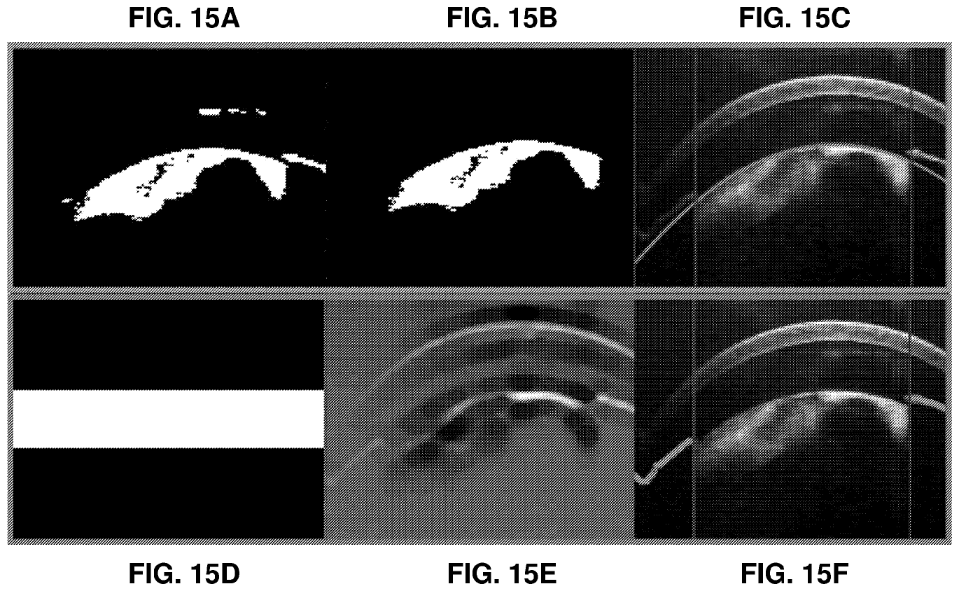

Additional methods other than those described supra to align the lateral center and z-axis of the TRF and ERF were tested. FIGS. 15A-15F illustrate the additional image processing steps used by these methods. The red box identifies steps related to the lens of the eye, and the blue box for steps related to the iris of the eye.

First, the B-scan was binarized and the cornea was segmented as detailed supra. In the binarized B-scan, components spatially connected to the corneal top surface were removed to form a binarized image of the lens and part of the iris (FIG. 15A). In the resulting binarized image, connected regions with an axial length greater than 40 μm were kept while the other regions were removed. The largest remaining component was considered as the lens (FIG. 15B). A polynomial was fit to the top surface of the lens between its left and right edges (FIG. 15C).

To identify the boundaries of the iris, a filter was designed to amplify the signal of the iris in the vis-OCT images (FIG. 15D). The filter took the signal amplitude of 3 axially adjacent pixels and subtracts the signal amplitude of the 8 pixels vertically adjacent to this region in the upwards and downwards direction. In vis-OCT, the iris appears as a thin surface owing to the attenuation of visible light by the pigmentation of the iris. The previewed B-scan was filtered using this filter (FIG. 15E) and the pixel of maximum intensity for each a-line not within the border of the lens was considered the axial position of the iris (FIG. 15F).

### Evaluation of Methods to Identify Lateral Position of Central Cornea

The following image processing schemes for matching the transverse origin of the ERF and TRF were tested with the image processing steps described supra.

1) X Center of mass: Let I(x,y,z) be the signal amplitude of the B-scan at depth z and lateral position x. the x center of mass is defined as:

$${COM}_{x} = \frac{{\sum }_{z} {\sum }_{x} I ( x , y , z ) x}{{\sum }_{z} {\sum }_{x} I ( x , y , z )}$$

The x center of mass was considered to be the origin of the ERF in the transverse direction.

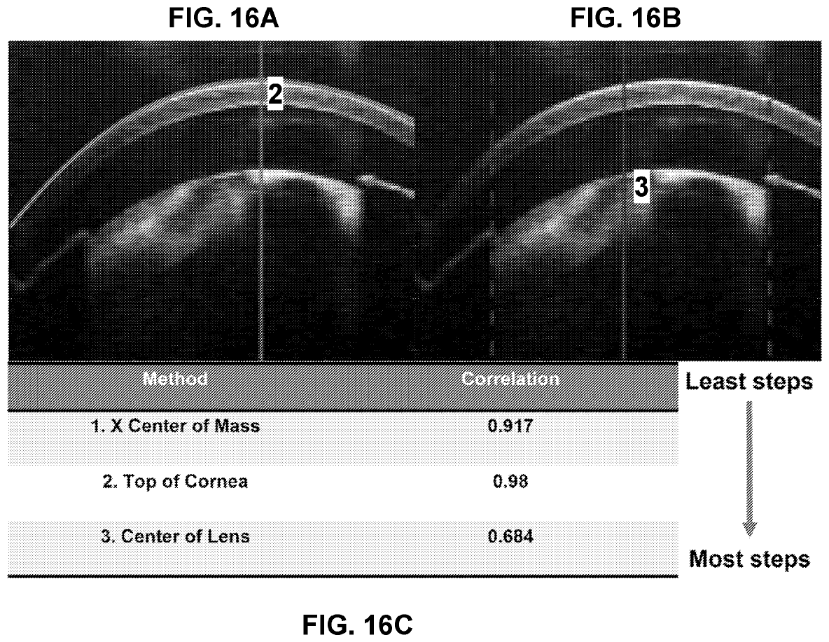

2) Top of the cornea: After fitting a polynomial to the top of the cornea, the top of the fitted cornea was considered to be the origin of the ERF (FIG. 16A).

3) Center of lens: After determining the boundaries of the lens, the midpoint between the lens boundaries was taken to be the origin of the ERF (FIG. 16B).

To evaluate the accuracy of each method, the transverse center of the central cornea was manually selected and selected positions were manually correlated with the automatically calculated position. The correlation of the methods with the manually selected transverse center was calculated and the methods were ordered from the fewest to most steps required (FIG. 16C). Overall, the top of the cornea method had the greatest correlation.

### Evaluation of Methods to Identify Optical Axis of Eye

The following methods for aligning the z-axes of the ERF and TRF were tested. When the z-axes of the ERF and TRF are misaligned, the orientation of the iris is tilted from the left and right side of the B-scan.

1) Y Center of Mass Slope: As detailed previously, the center of mass for the center B-scan (y^{th} B-scan) in the axial direction was calculated as a function of transverse position x:

$${COM}_{y} ( x ) = \frac{{\sum }_{z} I ( x , y , z ) x}{{\sum }_{z} I ( x , y , z )}$$

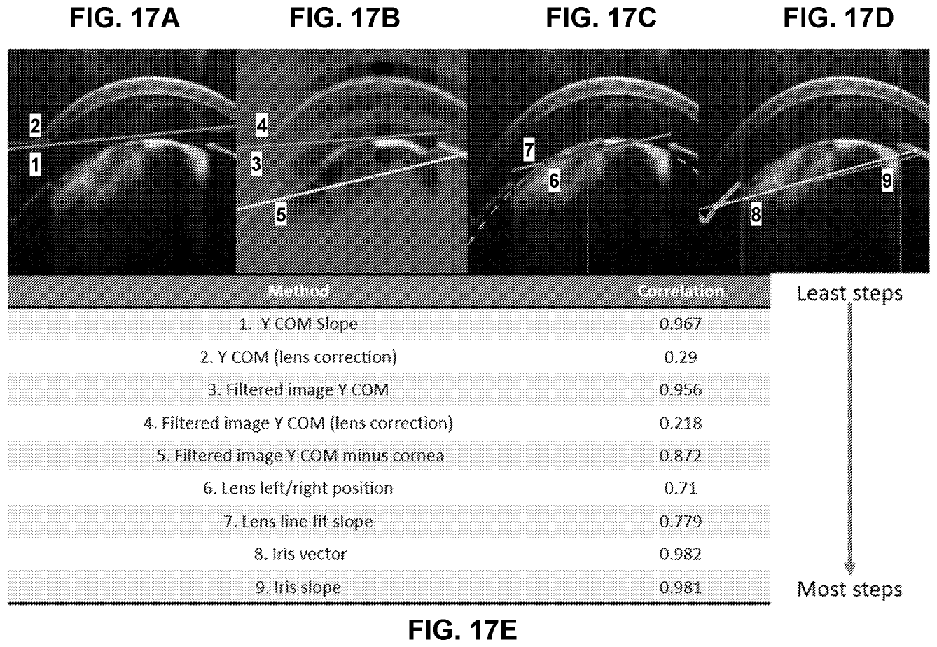

The slope of a line fit to COM_{y}(x) (FIG. 17A) was used as a metric assessing how misaligned the z-axes were.

2) Y Center of Mass Slope with Lens correction: This method is identical to Y Center of Mass Slope except only a-lines outside the lateral boundaries of the lens were considered.

3) Filtered Image Y Center of Mass Slope: This method is identical to Y Center of Mass Slope except that the input image is the image filtered by the custom ridge filter (FIG. 17B). An example of the filtered image is shown in FIG. 15E.

4) Filtered Image Y Center of Mass Slope with Lens Correction: A line is fit through COM_{y}(x) for the filtered image with a-lines corresponding to the lens removed. The fit slope was calculated.

5) Filtered Image Y Center of Mass Slope with Cornea remove: A line is fit through COM_{y}(x) for the filtered image with the signal corresponding to the cornea removed the same way as done for FIG. 14A.

6) Lens left/right position: The top position of the binarized lens on the left and right side as was determined. The magnitude of the difference of the top position of the left and right sides of the lens was computed (FIG. 17C).

7) Lens line fit slope: The slope of a line fit to the top position of the binarized lens from left to right was calculated.

8) Iris vector: The average depth position of the iris on the left and right side of the lens found and the magnitude of the difference was taken (FIG. 17D).

9) Iris slope: The slope of a line fit to the iris position from left to right was fitted.

To evaluate the accuracy of each scheme, a user manually drew a line normal to the optical axis of the eye and measured the correlation of each method with the angle of the manually drawn line. The y center of mass slope, iris vector, and iris slope were found to have the highest correlations (FIG. 17E). Since the y center of mass slope involved the least steps, making it more efficient for real-time processing, that method was used for determining the optical axis of the eye in the robotic AS-OCT system.

### Characterization of Optical Distortions

To assess how optical distortions influence 3D reconstruction, the chromatic and radial distortions of the robotic vis-OCT were assessed.

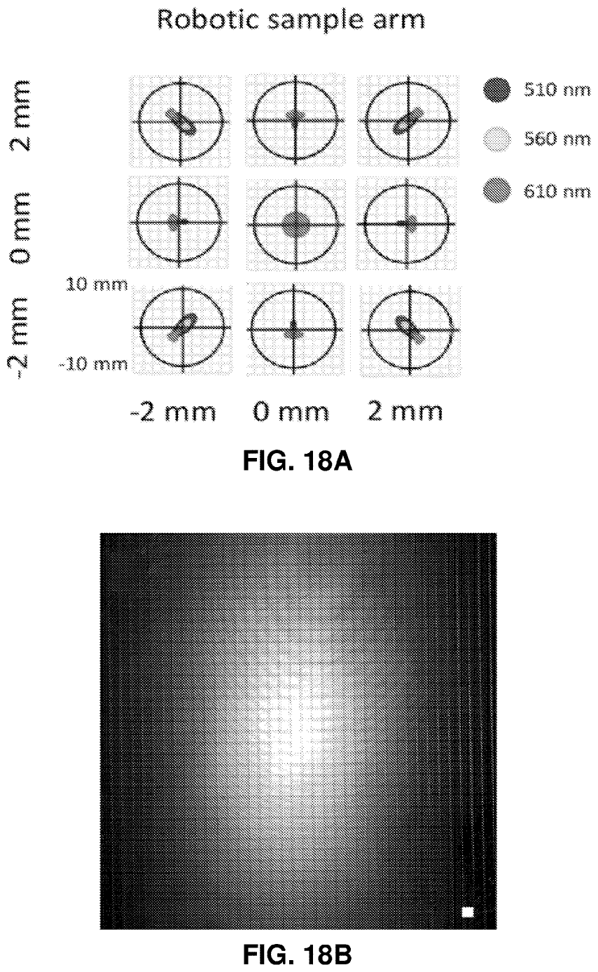

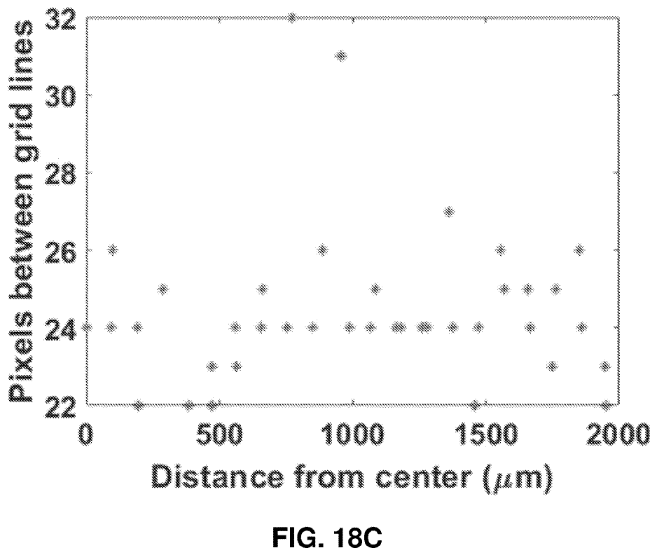

To measure radial distortions, a grid distortion target (Thorlabs R1L3S3PR) was imaged. The spacing between each grid was 100 μm. The distortion target was imaged using vis-OCT (FIG. 18B) and the spacing between grid points was measured as a function of distance away from the origin of the OCT image plane (FIG. 18C). A linear fit of the number of lateral pixels as a function of distance from the origin was # of pixels=0.0005 (m from center)+24.29, with the slope not statistically different from zero with a p-value of 0.822. Thus, the lateral pixel size does not change depending on the distance of the pixel from the center of the OCT field of view. Consequently, radial distortion was found to have minimal impact on the reconstructed volume. As the lateral dimension of the imaged grid distortion target was four times larger than what was used to acquire each volume of outflow pathway, correction of radial was not necessary for the robotic vis-OCT.

### Validation of Resolution

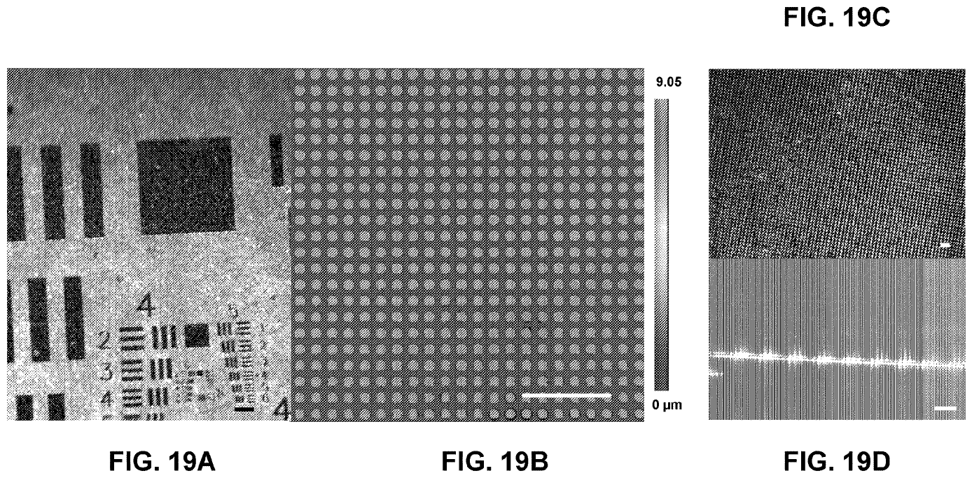

Using the radius of the airy disk definition, the theoretical lateral resolution of the robotic vis-OCT was calculated to be 9.4 μm. Lateral resolution was validated experimentally with a USAF51 target card. The smallest feature on the target card that could be resolved was group 5, element 6 (FIG. 19A), which has a line width of 8.77 μm. The size of the theoretical lateral resolution and resolution as measured by the USAF51 target card match within 1 μm. The theoretical axial resolution of vis-OCT is approximately 2 μm in air.

The resolution of vis-OCT by was validated by imaging a customized microfabricated polydimethylsiloxane phantom. The phantom consisted of cylindrical columns distributed in a uniformly spaced pattern. The structure of the phantom was characterized using a three-dimensional optical profiler (NexView, Zygo Corp). Each cylindrical column was 9.0 μm in height and 11.5 μm in diameter. The gap between columns was 8.5 μm (FIG. 19B). Using vis-OCT, the phantom was imaged (FIG. 19C). The columns and spacing in between the columns were clearly visualized, indicating that the vis-OCT had sufficient resolution to discern the 8.5 μm gap between the columns. Measurement of the column diameter and space between columns was 11.5 and 8.5 μm using vis-OCT, in agreement with the optical profiler. To assess the height of the column, a cross-sectional B-scan of the phantom (FIG. 19D) was examined. The height of the column was measured as 8.7 μm, slightly under the value recorded using the optical profiler.

Repeatability of Robotic as-OCT Positioning

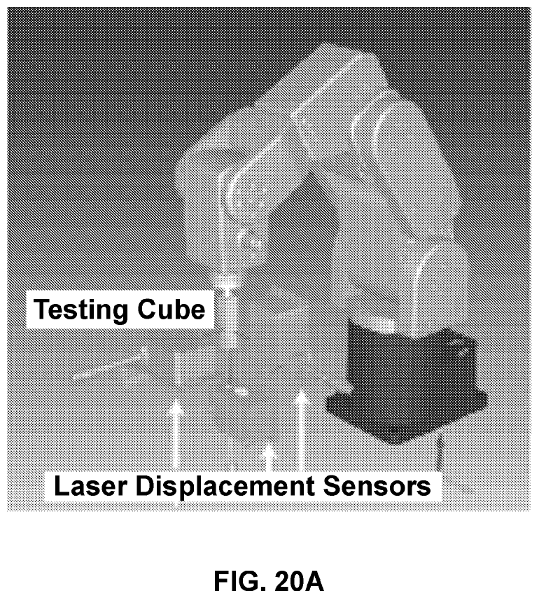

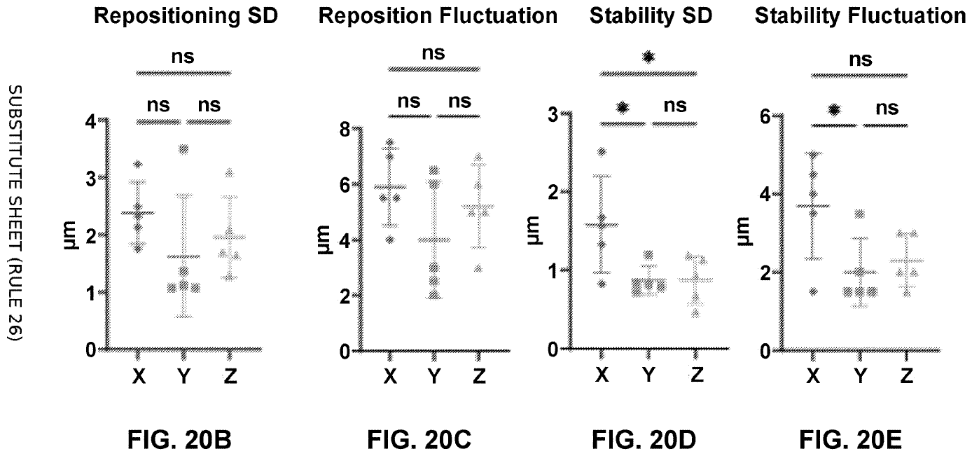

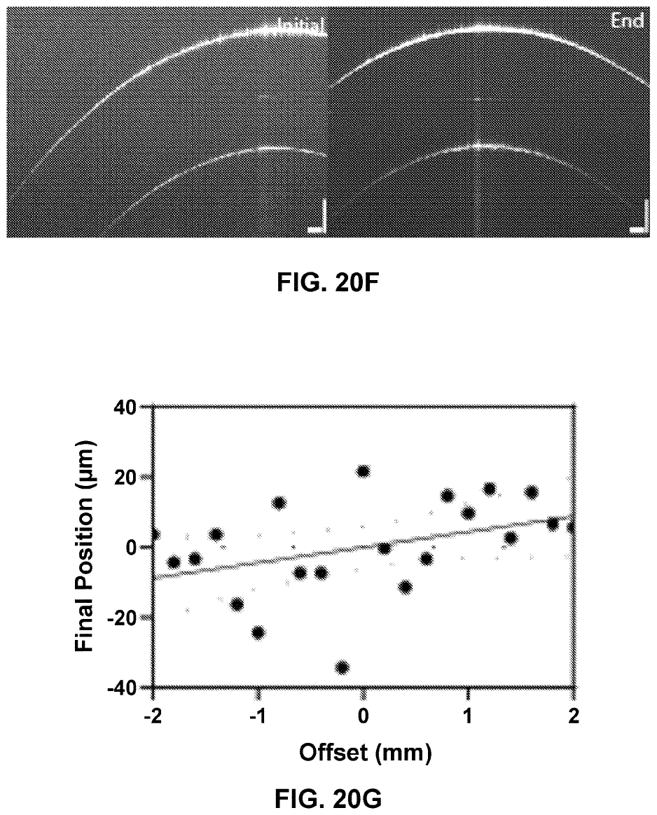

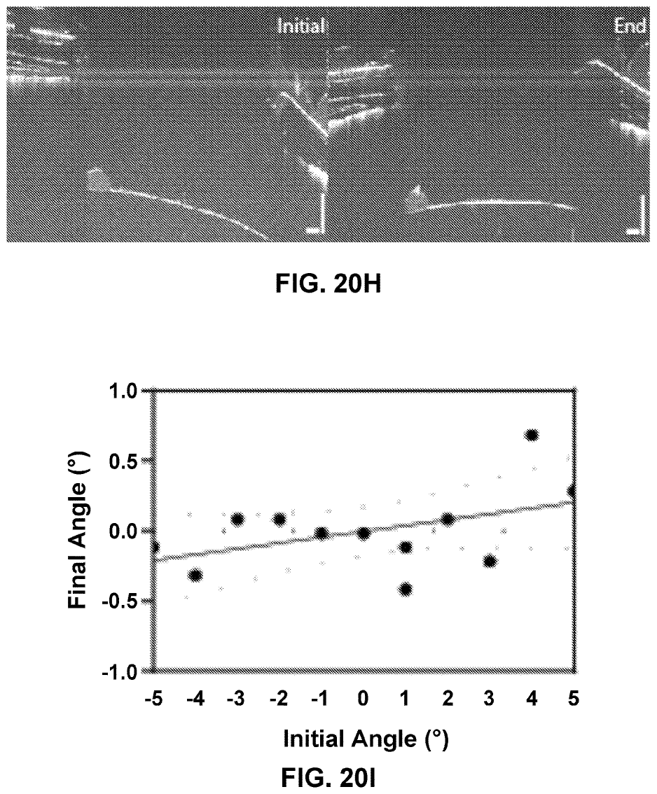

To monitor dynamic changes in structure, it is desirable that the positioning of the robot arm is repeatable over time and stable when held at a fixed position. To test the precision of the robot arm positioning, a test cube was attached to the robotic arm and the position of that test cube was tracked with laser displacement sensors in 3 orthogonal directions (FIG. 20A). To measure repeatability, the robotic arm was moved to the same position 200 times. When commanded to return to the same position 200 times for five different positions, the standard deviations in measured positions in the x, y, and z directions was 2.4, 1.6, and 1.9 μm respectively (FIG. 20B). The fluctuation in positions, defined as half the max range of positions, was 5.9, 4.0, and 5.2 μm in the x, y, and z axis respectively as shown in (FIG. 20C). To measure stability, the robotic arm was fixed in place and position measurements were taken every 2 seconds for a period of 400 seconds for five different positions. When held in place, the standard deviation of position in the x, y, and z directions were 1.6, 0.9, and 0.9 μm (FIG. 20D) with a maximum fluctuation of 3.7, 2.0, and 2.3 μm respectively (FIG. 20E).

Next, the precision of the lateral center finding and optical axis determination methods were tested for the ERF used in data acquisition. To quantify the precision of the methods, they were applied to a ROWE anterior segment phantom eye model. For testing the centering method, the cornea was displaced a distance between −2 mm to 2 mm from the center of the eye phantom in increments of 0.2 mm. The method to center the cornea was subsequently used and the recorded internal robot arm position was taken once the robot arm had achieved a stable position (FIG. 20F). FIG. 20G shows a linear fit of a final position of the robotic arm as a function of distance from the origin. The slope of the linear fit was not statistically significant different from zero although the fitted slope was 4.4 Mm/mm. The precision of the centering algorithm, defined as the standard deviation in final measured positions, was 13.9 μm.

For testing the optical axis alignment method, the phantom eye and TRF z-axes were misaligned between −5 to 5 degrees in increments of 1 degree. The algorithm to correct for the misaligned axes was subsequently used and final alignment once the robot arm had achieved a stable position was recorded (FIG. 20H). FIG. 20I shows a linear fit of final angular misalignment as a function of initial angular misalignment. The slope of the linear fit was not statistically significant different from zero although the fitted slope was 0.026 degree change in final angular misalignment per 1 degree change in initial angular misalignment. The precision of the z-axes alignment algorithm, defined as the standard deviation of final angular misalignment, was 0.28 degrees.

### Repeatable Positioning Enables Analysis of Dynamic Changes

A primary advantage of in vivo imaging over histology is the capability to assess functional properties of tissue. To assess the dynamic response of SC to external modulation, the full conventional outflow pathway in a C57BL/6 mouse was imaged before and 15 minutes after 1% pilocarpine administration. Pilocarpine is expected to increase SC size by constructing the pupil and pulling on the zonule fibers connecting the TM to the lens. The pull of the TM outwards expands SC size.

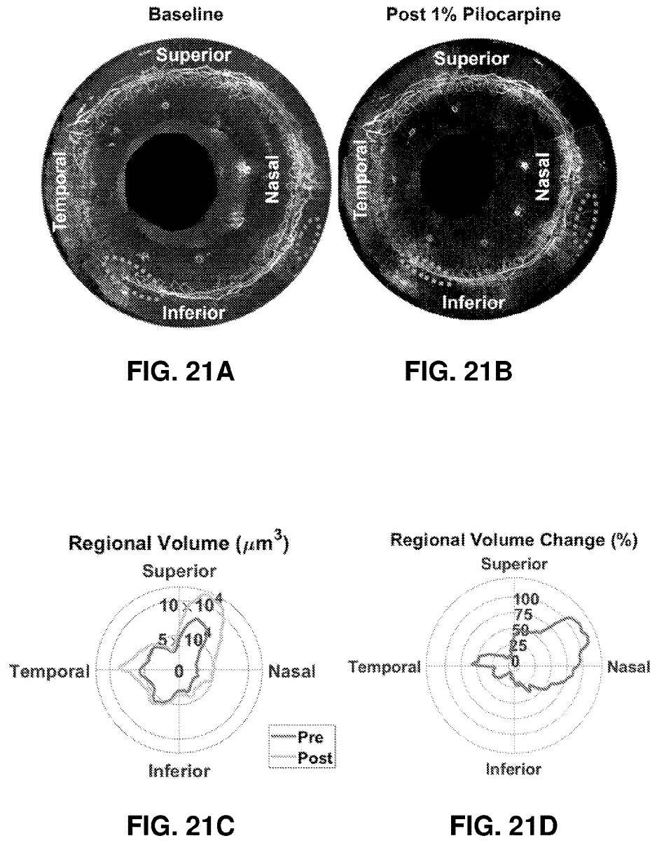

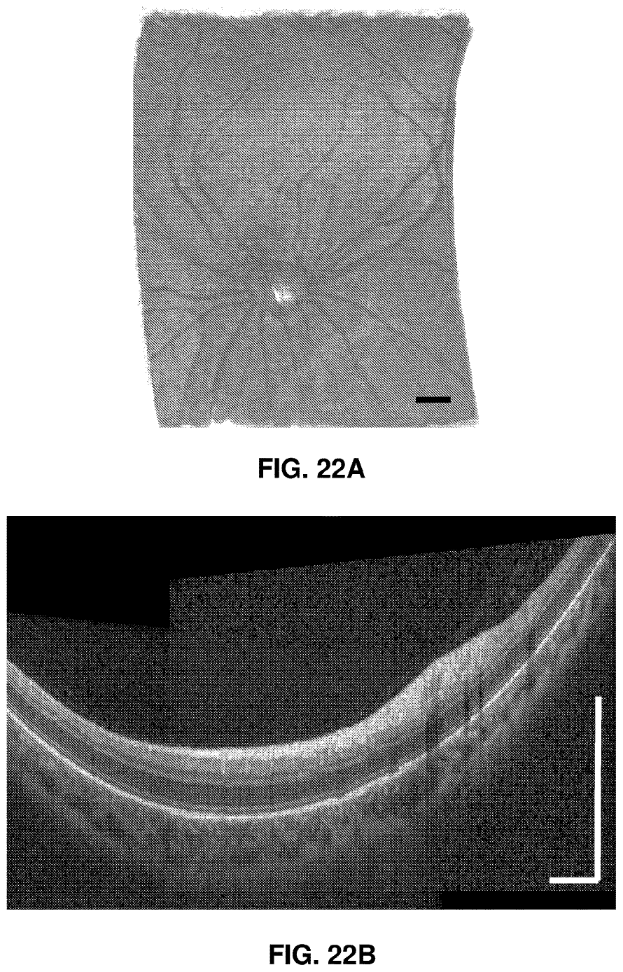

Consistent with pilocarpines mechanism of action, the pupil constricted (FIGS. 21A-21B). The outer edges of the lens are easily observed in the OCTA projection before pilocarpine administration and not 15 minutes after administration. Increased OCTA signal was observed in the nasal-superior region and decreased signal was observed in the temporal-inferior region. The green dotted region shows an area with decreased OCTA signal after administration and the red dotted area shows a region where a new vessel branch is observed. These patterns were consistent with regional changes in SC volume following pilocarpine administration, where SC volume per degree of eye increased the most in the nasal-superior region and least in the temporal-inferior region (FIG. 21C). Volume per degree of the eye was found by computing the number of voxels within one azimuthal degree of the final reconstructed volume. Volume values were averaged over a region of 20 degrees for stable readings. For this mouse, the volume increase per degree of the eye following pilocarpine administration was found to be 40.5±30.8% and ranged from 3.2 to 116.7%. Overall, the total volume increased by 15.5% in the inferior quadrant, 84.4% in the nasal quadrant, 39.7% in the superior quadrant, and 26.7% in the temporal quadrant. Just as with SC size, a segmental variation in volume changes was found following pilocarpine administration.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that components and methods other than those specifically exemplified can be employed in the practice of the invention without resort to undue experimentation. All art-known functional equivalents, of any such components and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for constructing a compound volumetric image of an object to be imaged from a plurality of optical coherence tomography (OCT) volumetric images acquired by an OCT imaging system, the method comprising:
selecting, from the plurality of OCT volumetric images, one or more overlapping pairs of OCT volumetric images, wherein each overlapping pair of OCT volumetric images includes a first OCT volumetric image corresponding to a first field of view (FOV) and a second OCT volumetric image corresponding to a second FOV, wherein the first FOV and the second FOV overlap in an overlap volume;
determining, for each respective selected overlapping pair of OCT volumetric images, a geometric transformation from the second FOV to the first FOV by:
identifying, in the first OCT volumetric image, one or more landmark volumes located in the overlap volume;
identifying, in the second OCT volumetric image, the one or more landmark volumes located in the overlap volume;
determining, based on a location of one or more landmark volumes in the first OCT volumetric image and a location of the one or more landmark volumes in the second OCT volumetric image, a mapping of the one or more landmark volumes in the second OCT volumetric image to the one or more landmark volumes in the first OCT volumetric image; and
determining, based on the mapping, the geometric transformation from the second FOV to the first FOV; and
constructing the compound volumetric image by applying the one or more geometric transformations of the one or more selected overlapping pairs of OCT volumetric images to merge the plurality of OCT volumetric images.

2. The method according to claim 1, wherein the determining, from the plurality of OCT volumetric images, the one or more overlapping pairs of OCT volumetric images comprises, for each of the one or more overlapping pairs of OCT volumetric images:
comparing a first position of the OCT imaging system corresponding to the first FOV and a second position of the OCT imaging system corresponding to the second FOV; and
determining, based on the comparing, that the first FOV and the second FOV overlap.

3. The method according to claim 2, wherein the first position of the OCT imaging system is defined by a tool reference frame (TRF) corresponding to a first position of a robot carrying a sample arm of the OCT imaging system and the second position of the OCT imaging system is defined by a TRF corresponding to a second position of the robot.

4. The method according to any of the preceding claims, wherein the determining, for each respective selected overlapping pair of OCT volumetric images, the geometric transformation from the second FOV to the first FOV further comprises:
generating a first point cloud representing one or more surfaces of the one or more landmark volumes in the first OCT volumetric image; and
generating a second point cloud representing one or more surfaces of the one or more landmark volumes in the second OCT volumetric image.

5. The method according to claim 4, wherein the determining, based on the location of the one or more landmark volumes in the first OCT volumetric image and the location of the one or more landmark volumes in the second OCT volumetric image, the mapping of the one or more landmark volumes in the second OCT volumetric image to the one or more landmark volumes in the first OCT volumetric image comprises:
calculating a mapping of points of the second point cloud to corresponding points of the first point cloud, wherein the calculated mapping minimizes a distance between the first point cloud and the second point cloud.

6. The method according to any of the preceding claims, wherein the identifying, in the first OCT volumetric image, the one or more landmark volumes located in the overlap volume comprises identifying, in the first OCT volumetric image, at least three common features of the object to be imaged that are visible in both the first OCT volumetric image and the second OCT volumetric image, and
wherein the identifying, in the second OCT volumetric image, the one or more landmark volumes located in the overlap volume comprises identifying, in the second OCT volumetric image, at least three common features of the object to be imaged that are visible in both the first OCT volumetric image and the second OCT volumetric image.

7. The method according to claim 6, wherein the first point cloud represents outer surfaces of the at least three common features in the first OCT volumetric image, and
wherein the second point cloud represents outer surfaces of the at least three common features in the second OCT volumetric image.

8. The method according to claim 6, wherein the first point cloud represents full volumes of the at least three common features in the first OCT volumetric image, and
wherein the second point cloud represents full volumes of the at least three common features in the second OCT volumetric image.

9. The method according to any of the preceding claims, further comprising, for each respective selected overlapping pair of OCT volumetric images, defining, for the first OCT volumetric image, a first tool reference frame (TRF), and defining, for the second OCT volumetric image, a second TRF, and
wherein the geometric transformation from the second FOV to the first FOV provides a pose of the second TRF relative to the first TRF.

10. The method according to claim 9, further comprising defining an object reference frame (ORF) providing world coordinates of the object being imaged.

11. The method according to claim 10, wherein a pose of the first TRF and a pose of the second TRF relative to the ORF are selected based on at least one characteristic of the object to be imaged in the ORF.

12. The method according to any of the preceding claims, wherein the constructing the compound volumetric image by applying the one or more geometric transformations of the one or more selected overlapping pairs of OCT volumetric images to merge the plurality of OCT volumetric images comprises, for at least one of the selected overlapping pairs of OCT volumetric images, transforming the second OCT volumetric image to a first tool reference frame (TRF) of the first volumetric image, and thereafter, applying a transformation mapping the first TRF to an object reference frame (ORF) to the transformed second OCT volumetric image.

13. The method according to any of the preceding claims, wherein the constructing the compound volumetric image by applying the one or more geometric transformations of the one or more selected overlapping pairs of OCT volumetric images to merge the plurality of OCT volumetric images comprises, transforming the plurality of OCT volumetric images to a single respective reference frame defined by the object reference frame (ORF).

14. The method according to any of the preceding claims, wherein an image characteristic is used to determine an origin and axes of a reference frame of the compound volumetric image, wherein the image characteristic is selected from the group consisting of an axis of symmetry, a region of interest, and a normal vector to a surface of a region of interest in the compound volumetric image.

15. The method according to any of the preceding claims, wherein the constructing the compound volumetric image by applying the one or more geometric transformations of the one or more selected overlapping pairs of OCT volumetric images to merge the plurality of OCT volumetric images comprises:
detecting errors, resulting from a loop closure problem, in an initial compound volumetric image formed by applying the one or more geometric transformations of the one or more selected overlapping pairs of OCT volumetric images;
recalculating, using the detected errors, the one or more geometric transformations to provide recalculated geometric transformations; and
applying the recalculated geometric transformations to merge the plurality of OCT volumetric images to construct the compound volumetric image.

16. The method according to claim 15, wherein the recalculated geometric transformations map each of the plurality of OCT volumetric images, each of which corresponds to a respective tool reference frame (TRF), to an object reference frame (ORF) to address the loop closure problem whereby successive transformations lead to cumulative errors in the compound volume.

17. The method according to claim 16, wherein each respective recalculated geometric transformation maps a respective OCT volumetric image to the ORF to minimize a distance between respective landmark volume point clouds and all other point clouds.

18. The method according to any of the preceding claims, wherein the plurality of OCT volumetric images are acquired by an OCT imaging system having a sample arm configured to move with multiple translational and rotational degrees of freedom.

19. The method according to claim 18, wherein the sample arm is carried by a robotic arm.

20. The method according to claim 19, wherein the first FOV is determined by a first position of the robotic arm, and
wherein the second FOV is determined by a second position of the robotic arm.

21. The method according to any of the preceding claims, wherein the compound volumetric image has a compound imaging volume (CIV) that is enlarged relative to an imaging volume of each respective OCT volumetric image of the plurality of OCT volumetric images.

22. The method according to any of the preceding claims, wherein each of the plurality of OCT volumetric images is captured from a location of the OCT imaging system at which an image plane of the OCT imaging system is approximately tangential to a surface of the object to be imaged.

23. The method according to any of the preceding claims, wherein a primary symmetry axis within each cross-sectional B-scan of each of the plurality of OCT volumetric images is aligned with a normal vector of a surface of the object to be image.

24. The method according to any of the preceding claims, wherein deviations in a center of mass in a z-axis of each respective tool reference frame (TRF) defined for a respective OCT volumetric image is minimized from both the left and right sides of a cross-sectional image.

25. The method according to any of the preceding claims, wherein the object to be imaged is an eye, and wherein, for an anterior segment of the eye, the reference frame of the eye is defined as the object reference frame (ORF), wherein axes of the ORF are defined using an optical axis of a respective tool reference frame (TRF) defined for a respective OCT volumetric image and parallelizing the optical axis of the respective TRF with an optical axis of the eye, the optical axis of the eye being parallel to a normal vector to a central cornea of the eye.

26. The method according claim 25, wherein the optical axis of a respective TRF is centered on the cornea by centering a point on the cornea closest to the OCT imaging system at a center of a respective corresponding OCT field of view,
wherein centering the OCT FOV on top of the cornea defines the origin of the ORF.

27. The method according to claim 25 or claim 26, wherein, after identifying the optical axis and center of the eye, a predefined circular trajectory relative to the optical axis and eye center that is perpendicular to the normal vector of the eye along the scan trajectory is mapped out.

28. A system for constructing an aggregate volumetric image of an object to be imaged from a plurality of optical coherence tomography (OCT) volumetric images, the system comprising:
an OCT imaging system, comprising:
a light source,
a sample arm integrated with a robotic arm configured to shift a field of view (FOV) of the OCT imaging system,
a reference arm,
a balanced spectrometer configured to detect interference signals in light reflected from the sample arm and light reflected from the reference arm, and
processing circuitry configured to:
construct, based on data provided by the balanced spectrometer, a plurality of OCT volumetric images, and
perform the method according to any of the preceding claims.

29. A non-transitory computer readable medium having stored thereon processor executable instructions that, when executed by one or more processors, cause the one or more processors to perform the method according to any of claims 1 through 27.

30. A method for determining a plurality of image acquisition positions for an OCT imaging system during imaging of an object to be imaged by the OCT imaging system, each image acquisition position corresponding to a field of view (FOV), the method comprising:
defining an object reference frame;
representing, in the object reference frame, a geometry of a surface of the object to be imaged;
defining an imaging reference frame having a first axis corresponding to an axial direction of an OCT beam of the OCT imaging system and second and third axes corresponding to scanning directions of a beam scanner of the OCT imaging system, wherein the imaging reference frame is movable relative to the object reference frame by motion of the OCT imaging system;
determining a rough positional relationship between the object reference frame and the OCT imaging system;
determining, based on the geometry of the surface of the object to be imaged in the object reference frame and the rough positional relationship between the object reference frame and the OCT imaging system, the plurality of image acquisition positions for the OCT sample arm,
wherein, for each respective image acquisition position of the plurality of image acquisition positions for the OCT imaging system, the imaging reference frame is arranged such that the second and third axes form a plane that is approximately tangential to the geometry of the surface of the object in the object reference frame.

31. The method according to claim 30, wherein the plurality of image acquisition positions for the OCT imaging system are determined such that each respective FOV corresponding to a respective image acquisition position overlaps with at least one other respective FOV corresponding to at least one other respective image acquisition position.

32. The method according to claim 30 or 31, wherein the defining the imaging reference frame comprises defining an origin of the imaging reference frame as a focal point of an incident beam of the OCT imaging system.

33. The method according to any of claims 30 through 32, further comprising controlling the OCT imaging system to move from a first of the plurality of image acquisition positions to a last of the plurality of image acquisition positions through each of the remaining image acquisition positions and to acquire, at each respective image acquisition positions of the plurality of image acquisition positions, an OCT volumetric image.

34. The method according to claim 33, wherein controlling the OCT imaging system to move from a first of the plurality of image acquisition positions to a last of the plurality of image acquisition positions through each of the remaining image acquisition positions comprises providing instructions to a robotic arm, with which a sample arm of the OCT imaging system is integrated, to assume a plurality of positions that correspond to the plurality of image acquisition positions.

35. The method according to any of claims 30 through 34, wherein the representing, in the object reference frame, a geometry of a surface of the object to be imaged comprises identifying, in one or more OCT volumetric images obtained by the OCT imaging system, the object to be imaged or a feature of the object to be imaged.

36. The method according to claim 35, wherein the determining the rough positional relationship between the object reference frame and the OCT imaging system comprises:
pre-calibrating, based on the identification of the object to be imaged or the feature of the object to be imaged, the relationship between the object reference frame and the OCT imaging system; and
calibrating, based on a plurality of additional OCT volumetric images obtained by the OCT imaging system, the relationship between the object reference frame and the OCT imaging system.

37. A system for acquiring a plurality of OCT volumetric images from a plurality of image acquisition positions, the system comprising:
an OCT imaging system, comprising:
a light source,
a sample arm integrated with a robotic arm configured to shift a field of view (FOV) of the OCT imaging system,
a reference arm,
a balanced spectrometer configured to detect interference signals in light reflected from the sample arm and light reflected from the reference arm, and
processing circuitry configured to perform a method according to any of claims 30-36 for determining the plurality of image acquisition positions, each image acquisition position corresponding to a field of view (FOV) of the OCT imaging system.

38. The system according to claim 37, wherein the processor is further configured to perform the method according to any of claims 1 through 27.

39. A non-transitory computer readable medium having stored thereon processor executable instructions that, when executed by one or more processors, cause the one or more processors to perform the method according to any of claims 30 through 36.

40. The non-transitory computer readable medium according to claim 39, having further stored thereon processor executable instructions that, when executed by the one or more processors or by one or more additional processors, cause the one or more processors and/or the one or more additional processors to perform the method according to any of claims 1 through 27.

41. An optical coherence tomography (OCT) system, comprising:
a light source;
a reference arm;
a sample arm mounted on a robotic arm, the robotic arm configured to move the sample arm to a plurality of image acquisition positions to provide for freeform OCT imaging, each respective image acquisition position corresponding a respective field of view (FOV).

42. The OCT system according to claim 41, wherein the robotic arm is a six degree-of-freedom (6DoF) robotic arm.

43. The OCT system according to claim 41 or 42, further comprising a balanced spectrometer configured to detect interference signals in light reflected from the sample arm and light reflected from the reference arm, and
processing circuitry configured to construct, based on data provided by the balanced spectrometer, a plurality of OCT volumetric images.

44. The OCT system according to any of claims 41 through 43, further comprising a processor configured to perform the method according to any of claims 1 through 27.

45. The OCT system according to any of claims 41 through 44, further comprising a processor configured to perform the method according to any of claims 30 through 36.
